(19) United States
(12) Patent Application Publication
(10) Pub. No.: US 2024/0417806 A1
(43) Pub. Date: Dec. 19, 2024

(54) METHODS OF IDENTIFYING AND TREATING CANCERS UTILIZING ALTERNATIVE LENGTHENING OF TELOMERE PATHWAY (ALT+ CANCERS)

(71) Applicants: Drexel University, Philadelphia, PA (US); New York Institute of Technology, Old Westbury, NY (US)

(72) Inventors: Ming Xiao, Huntingdon Valley, PA (US); Dong Zhang, Bardonia, NY (US)

(21) Appl. No.: 18/742,888

(22) Filed: Jun. 13, 2024

Related U.S. Application Data
(60) Provisional application No. 63/472,889, filed on Jun. 14, 2023.

(51) Int. Cl. C12Q 1/6886; C12Q 1/683

(52) U.S. Cl. CPC C12Q 1/6886; C12Q 1/683; C12Q 2600/112

(57) ABSTRACT

Described herein is a method of categorizing a cancer (such as into TEL+ cancer or ALT+ cancer). The method includes performing a global analysis of telomere lengths in cancer cells of the cancer, and determining the category of the cancer based on patterns of telomere lengths. Also described herein is a method of treating cancer based on the categorizing of the cancer.

### CROSS-REFERENCE TO RELATED APPLICATIONS

The present application claims priority under 35 U.S.C. § 119(e) to U.S. Provisional Patent Application No. 63/472,889, filed Jun. 14, 2023, which is incorporated herein by reference in its entirety.

### STATEMENT REGARDING FEDERALLY SPONSORED RESEARCH

This invention was made with government support under 5R01HG005946 awarded by the NIH. The government has certain rights in the invention.

### SEQUENCE LISTING

The XML text file named “046528-7142US1(01065)_Seq Listing.xml” created on Jun. 10, 2024, comprising 5,584 bytes, is hereby incorporated by reference in its entirety.

### BACKGROUND

One of the key hallmarks of cancers is the replicative immortality. Maintaining a healthy length of telomeres in cancer cells is critical for the long-term survival of cancers. About 85% to 90% of cancers activate the expression of telomerase (TEL+) as the telomere maintenance mechanism (TMM), while about 10% to 15% of cancers utilize the homology-dependent repair-based Alternative Lengthening of Telomere (ALT+) pathway.

ALT+ cancers are more susceptible to certain treatments, such as the inhibition of FANCM, ATR, or PARP. Existing assays for identifying ALT+ cancers, however; have various limitations.

There is a need for reliable assays that can reliably identify ALT+ cancers. The present invention addresses this need.

### SUMMARY

In some aspects, the present invention is directed to the following non-limiting embodiments:

### Method of Categorizing Cancer

In some aspects, the present invention is directed to a method of categorizing a cancer.

In some embodiments, the method comprises: performing a global analysis of telomere lengths in cancer cells of the cancer.

In some embodiments, an increased abundance of fusions/internal telomere-like sequence (ITS+) in the cancer cells relative to a reference abundance indicates a cancer utilizing an alternative lengthening of telomere pathway (ALT+ cancer).

In some embodiments, an increased abundance of fusions/internal telomere-like sequence loss (ITS−) in the cancer cells relative to a reference abundance indicates an ALT+ cancer.

In some embodiments, an increased abundance of telomere-free ends (TFEs) in the cancer cells relative to a reference abundance indicates an ALT+ cancer.

In some embodiments, an increased abundance of telomeres having 20% longer length than a natural telomere length (super-long telomeres) in the cancer cells relative to a reference abundance indicates an ALT+ cancer.

In some embodiments, an increased level of heterogeneity of telomere lengths at the whole-genome level in the cancer cells relative to a reference level indicates an ALT+ cancer.

In some embodiments, performing the global analysis of telomere lengths in the cancer cells comprises: contacting genomic DNA of the cancer cells with a guide RNA (gRNA) having a portion complementary to a telomere-specific motif, and a nickase to introduce a nick in the telomere; contacting the nicked DNA with a polymerase and a nucleotide labeled with a first dye such that the nucleotide labeled by the first dye is incorporated into the nicked telomere; and detecting the telomere using a signal of the first dye.

In some embodiments, the gRNA guides the nikase to the telomere, thereby allowing the nickase to introduce the nick in the telomere.

In some embodiments, the gRNA is complementary to a telomere repeat (TTAGGG)n.

In some embodiments, the nikase is a Cas9 nickase.

In some embodiments, the method further comprises labeling the genomic DNA of the cancer cells with a second dye labeling the genomic DNA; and detecting the genomic DNA according to a signal of the second dye.

In some embodiments, the first dye or the second dye is a fluorescent dye.

In some embodiments, the method further comprises generating a recognition pattern on each chromosome of the cancer cells; and matching sections of the cancer cell chromosomes with chromosome sections of a healthy cell according to the recognition pattern.

In some embodiments, generating the recognition pattern the chromosomes of the cancer cells comprises: contacting the genomic DNA with a motif-specific nicking endonuclease, thereby producing a second nick in the genomic DNA at the motif sequence; and contacting the nicked DNA with a polymerase and a nucleotide labeled with a third dye such that the nucleotide labeled with the third dye is incorporated into the nicked DNA at the motif sequence location.

In some embodiments, the motif-specific nicking endonuclease is Nt.BspQI.

In some embodiments, the third dye is the same as the first dye or the third dye produce a same signal as the first dye.

In some embodiments, the third dye is different from the first dye or the third dye produce a different signal from that produced by the first dye.

In some embodiments, detecting the telomere according to the signal of the first dye comprises analyzing the genomic DNA labeled with the first dye according to a nanochannel array method.

### Method of Treating Cancer

In some aspects, the present invention is directed to a method of treating cancer in a subject in need thereof.

In some embodiments, the method comprises: determining whether the cancer is a cancer having activated expression of telomerase (TEL+ cancer) or a cancer utilizing an alternative lengthening of telomere pathway (ALT+ cancer); and if the cancer is determined to be an ALT+ cancer, administering to the subject an effective amount of an inhibitor for Fanconi anemia, complementation group M (FANCM), an inhibitor for ataxia telangiectasia and Rad3-related (ATR), or an inhibitor for poly (ADP-ribose) polymerase (PARP).

In some embodiments, determining whether the cancer a TEL+ cancer or an ALT+ cancer comprises performing a global analysis of telomere lengths in cancer cells of the cancer.

In some embodiments, an increased abundance of fusions/internal telomere-like sequence (ITS+) in the cancer cells relative to a reference abundance indicates an ALT+ cancer;

In some embodiments, an increased abundance of fusions/internal telomere-like sequence loss (ITS−) in the cancer cells relative to a reference abundance indicates an ALT+ cancer;

In some embodiments, an increased abundance of telomere-free ends (TFEs) in the cancer cells relative to a reference abundance indicates an ALT+ cancer;

In some embodiments, an increased abundance of telomeres having 20% longer length than a natural telomere length (super-long telomeres) in the cancer cells relative to a reference abundance indicates an ALT+ cancer; and/or

In some embodiments, an increased level of heterogeneity of telomere lengths at the whole-genome level in the cancer cells relative to a reference level indicates an ALT+ cancer.

In some embodiments, the inhibitor for FANCM is at least one selected from the group consisting of MM2 peptide, and an RNA interference molecule targeting FANCM.

In some embodiments, the inhibitor for ATR is at least one selected from the group consisting of ART0380, ATG-018, ATRN-119, AZ20, berzosertib, Camonsertib (RP-3500), ceralasertib, CGK 733, dactolisib, elimusertib, ETP-46464, HAMNO (NSC-111847), IMP9064, SKLB-197, Schisandrin B, Torin 2, Tuvusertib, VE-821, VX-803 (M4344), and an RNA interference molecule targeting ATR.

In some embodiments, the inhibitor for PARP is at least one selected from the group consisting of 3-aminobenzamide, CEP 9722, E7016, iniparib, niraparib, olaparib, pamiparib, rucaparib, talazoparib, veliparib, and an RNA interference molecule targeting PARP.

In some embodiments, performing the global analysis of telomere lengths in the cancer cells comprises: contacting genomic DNA of the cancer cells with a guide RNA (gRNA) having a portion complementary to a telomere-specific motif, and a nickase to introduce a nick in the telomere; contacting the nicked DNA with a polymerase and a nucleotide labeled with a first dye such that the nucleotide labeled by the first dye is incorporated into the nicked telomere; and detecting the telomere according to a signal of the first dye.

In some embodiments, the gRNA guides the nikase to the telomere, thereby allowing the nickase to introduce the nick in the telomere.

In some embodiments, the gRNA is complementary to a telomere repeat (TTAGGG)n.

In some embodiments, the nikase is a Cas9 nickase.

In some embodiments, the method further comprises labeling the genomic DNA of the cancer cells with a second dye labeling the genomic DNA; and detecting the genomic DNA according to a signal of the second dye.

In some embodiments, the first dye or the second dye is a fluorescent dye.

In some embodiments, the method further comprises generating a recognition pattern on each chromosome of the cancer cells; and matching sections of the cancer cell chromosomes with chromosome sections of a healthy cell according to the recognition pattern.

In some embodiments, generating the recognition pattern the chromosomes of the cancer cells comprises: contacting the genomic DNA with a motif-specific nicking endonuclease, thereby producing a second nick in the genomic DNA at the motif sequence; and contacting the nicked DNA with a polymerase and a nucleotide labeled with a third dye such that the nucleotide labeled with the third dye is incorporated into the nicked DNA at the motif sequence location.

In some embodiments, the motif-specific nicking endonuclease is Nt.BspQI.

In some embodiments, the third dye is the same as the first dye or the third dye produce a same signal as the first dye.

the third dye is different from the first dye or the third dye produce a different signal from that produced by the first dye.

In some embodiments, detecting the telomere according to the signal of the first dye comprises analyzing the genomic DNA labeled with the first dye according to a nanochannel array method.

In some embodiments, the subject is a mammal or a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 provides a schematic description of the single-molecule telomere length assay, in accordance with some embodiments. In both telomere labeling schemes, the Cas9 D10A (Cas9n) directed by the telomere gRNA nicks the telomere repeats (TTAGGG)n and Nt.BspQI globally nicks the GCTCTTC motif. In the two-color scheme, all nicks are labeled with green fluorescently labeled nucleotides. In the three-color scheme, the Nt.BspQI-generated nicks are labeled with green fluorescently labeled nucleotides, and the telomeres are labeled with red fluorescently labeled nucleotides. All samples are linearized in the nanochannels and optically imaged. De novo assembly is performed, and the telomere label intensities are used for calculating the telomere length. The sequences shown in FIG. 1 are listed below:

Sequence Motif, forward strand
(SEQ ID NO: 1)
ATGCTCTTCNGTGATC
Sequence Motif, reverse strand
(SEQ ID NO: 2)
GATCACNGAAGAGCAT
Telomere, forward strand
(SEQ ID NO: 3)
GGTTAGGGTTAGGGTTAGGGTTAGGGTTAGGGTTAGGG
Telomere, reverse strand
(SEQ ID NO: 4)
CCCTAACCCTAACCCTAACCCTAACCCTAACCCTAACC
gRNA, telomere complementary region
(SEQ ID NO: 5)
UUAGGGUUAGGGUUAGGGUU

FIGS. 2A-2C: global sequence motif-dependent nick-labeling distinguishes subtelomeres and identifies subtelomeric structural variants, in accordance with some embodiments. FIG. 2A: The subtelomere repeat elements of 1p, 5q, and 8p are shown as colored blocks, with each color indicating a paralogy block of segmentally duplicated subtelomeric repeat elements (SREs) as defined in Stong et al. (Genome Res 24: 1039-1050). The telomeres are designated as a red T. The consensus maps of nick-labeled single molecules after de novo assembly and alignment with the hg38 reference are shown as light blue bars; the dark blue vertical ticks within these bars indicate the Nt.BspQI nick-label sites. The single molecules that comprise the respective consensus map are the yellow lines shown directly below each map. The labels on the single molecules that align with the hg38 reference are dark green, and those that do not align are light blue. The dark blue and red bars designate the overlapping regions between the chromosome arms. FIG. 2B: The major haplotypes of Chromosome 6p detected from eight individuals. The hg38 6p subtelomere reference sequence is shown as a light blue bar; the dark blue vertical ticks within these bars indicate the in silico Nt.BspQI nick-label sites. The black line segment designates a telomere-adjacent gap in the hg38 reference sequence. Each haplotype is represented as a distinct consensus map of single molecules from the designated genome (yellow line) aligned to the hg38 6p reference sequence. The labels on these consensus maps that align with the hg38 reference are dark green, and those that do not align are light blue. FIG. 2C: The two distinct haplotypes of the cancer genome UMUC3 for Chromosome 6p.

FIGS. 3A-3C: Colabeling telomere (TTAGGG)n tracts using gRNA-directed CRISPR/Cas9 nickase and subtelomeres using global motif-dependent nickase Nt.BspQI, in accordance with some embodiments. The hg38 subtelomere reference sequences are shown as light blue bars; the dark blue vertical ticks within these bars indicate in silico Nt.BspQI nick-label sites. The black line segments designate telomere-adjacent gaps in the hg38 reference sequence. Individual single-molecule maps were de novo assembled into the consensus maps (yellow lines), which were then aligned with the hg38 reference (light blue bars). The green lines on the consensus maps are Nt.BspQI (GCTCTTC) sites that align to the corresponding reference site. Those that do not align with the reference are designated with light blue lines. Representative examples of raw images of single molecules that were used to create the consensus maps are shown. FIGS. 3A-3B: Three-color consensus maps and single molecules for Chromosomes 3p and 12p of sample GM11832. The DNA backbone is blue, labeled subtelomeric Nt.BspQI sites are green, and the telomere (TTAGGG)n tracts are red. FIG. 3C: Two-color consensus maps and representative images of single molecules of both haplotypes for Chromosome 2q of sample IMR90-PD17. The DNA backbone is blue, and both the labeled subtelomeric Nt.BspQI sites and the telomere (TTAGGG)n tracts are green.

FIG. 4: Single-telomere length analysis results for 8q. (Top) The hg38 subtelomere reference sequence for 8q is shown as a light blue bar; the dark blue vertical ticks within these bars indicate in silico Nt.BspQI nick-label sites. The black line segment designates a telomere-adjacent gap in the hg38 reference sequence. The green lines on the consensus maps are Nt.BspQI (GCTCTTC) sites that align to the corresponding reference site. Those that do not align with the reference are designated with light blue lines. Representative examples of raw images of single molecules from two-color labeling experiments for each of the samples that were used to create the consensus map are shown. The human diploid fibroblast cell line IMR90 was sampled at population doubling 17 (IMR90-PD17), at PD45 (IMR90-PD45), and at senescence (IMR90-senescence; PD61). The immortal telomerase-positive cancer cell lines UMUC3 (bladder cancer) and LNCaP (prostate cancer) were also sampled. (Bottom) Telomere (TTAGGG)n tract length results for 8q from each of these samples. Each single-molecule telomere length measurement for 8q is represented by a dot, and the average telomere length for 8q in each sample is shown as a horizontal line.

FIGS. 5A-5B: Telomere labeling identifies incorrectly represented and uncharacterized subtelomeres. FIG. 5A: The 0.55- to 0.8-megabase region of the hg38 subtelomere reference sequence for 1p of UMUC3 is shown as a light blue bar; the dark blue vertical ticks within these bars indicate in silico Nt.BspQI nick-label sites. The hg38 reference sequence continuing toward the putative 1p telomere is not shown. The green lines on the single-molecule maps are Nt.BspQI (GCTCTTC) sites. Representative examples of raw images of several of these single molecules from two-color labeling experiments are shown. The single DNA molecules used to form the consensus map for Chromosome 1p each align with the 0.6-Mb region of the hg38 reference, and all contain intense telomere end labels. FIG. 5B: Five telomere-containing consensus maps that could not be aligned with the hg38 reference sequence. Red bar designates an SRE located on 4p, 4q, and several acrocentric short-arm subtelomeres.

FIG. 6A shows typical fosmid molecules with 800 bp telomeres, green dots representing Nt.BspQI sequence motifs, and red dots for CRISPR/Cas9 telomeric labeling. The DNA backbone is blue. 99.3% of the fosmid molecules ware labeled at telomeric sites. The total raw intensity of telomeric labeling from individual fosmids is plotted in FIG. 6B. The telomere labeling intensity is following the laser power exponential decay. The intensity is the highest close to the center of the imaging frame, and the weakest at the edges. After normalizing the telomeric intensity to the center of the imaging frame according to the laser power exponential decay. Use the center of the intensity to represent the true 800 bp telomeric repeats, the average telomere length is calculated to be 814 bp±195 bp. The present study also tested the system with another fosmid of 100 bp telomeric repeats. 93.4% of fosmids are labeled at telomeric sites, the average telomere length is calculated to be 153 bp±97 bp. The shorter telomere length measurements show higher variation. Clearly, the method herein allows the detection of telomeric repeats as short as 100 bp. To make the calibration more accurate, it is necessary to have a standard of a few kb. However, it is impossible to stably clone several kb of telomere sequence. The present study decided to randomly select 4 molecules of the fosmids containing 800 bp telomere as a group to simulate a 3.2 kb telomere. The average telomere length is calculated to be 3056 bp±456 bp, which is in good agreement with expected results. The calibration is summarized in FIG. 6C.

FIG. 7: the assembly results for the IMR90 and UMUC cell lines. For these cell lines, the present study generate data of more than 60× coverage with and without Cas9 telomeric labeling. The consensus maps from de novo assembly and the hg38 reference are shown as light blue bars and the dark blue vertical lines within these indicate the Nt.BspQI nick-label sites. The single molecules that comprise the respective consensus map are the yellow lines shown directly below the reference and consensus maps. The labels on the single molecules that align with the hg38 reference are dark green and those that do not align are light blue. The top consensus map were nick-labeled with Nt.BspQI and Cas9. The telomeres are the additional labels on the far left side of the single molecules (red T above) which are absent in the sample without Cas9. This clearly shows that Cas9 will specifically label the telomeric sites.

FIG. 8: 32 molecules that were measured for 12p of LNCaP. The telomere length was measured for approximately 30 molecules for each chromosome arm of a sample. The telomeres are designated as a red T. The consensus map aligned to the hg38 reference is shown above the nick-labeled single molecules after de novo assembly.

FIGS. 9A-9E illustrate certain aspects of a variety of telomere and chromosome end features that are detected by the SMTA-OM, in accordance with some embodiments. FIG. 9A: Chromosome ends with highly variable sizes of telomeres of the chromosome arm 2q. FIG. 9B: Extrachromosomal telomeric repeats (ECTRs). FIG. 9C: Chromosome ends without detectable telomere signals, or telomere-free ends (TFEs) of the chromosome arm 15q. FIG. 9D: A fusion molecule with internal telomere-like sequence (Fusion/ITS+) derived from the chromosome arm 5q. FIG. 9E: Two fusion molecules without internal telomere-like sequence (Fusion/ITS−) of the chromosome arm 21q. “T” in red font indicates the telomere location of a specific hg38 chromosome arm. The discrete and less intense green labels on a DNA molecule were used to align with the consensus map and assist in assigning of the DNA molecule to a specific chromosome arm.

FIGS. 10A-10B illustrate certain aspects of the analysis of the end telomere (−TFE) mean lengths and standard deviations from U2OS (green), SK-MEL-2 (red), Saos-2 (gray), UMUC3 (yellow), LNCaP (blue), and IMR90-S(purple), in accordance with some embodiments. FIG. 10A: End telomere (−TFE) mean lengths from all the p chromosome arms that were detected by the SMTA-OM. FIG. 10B: End telomere (−TFE) mean lengths from all q chromosome arms that were detected by the SMTA-OM. Chromosome arms that are not depicted did not have measurements from at least one ALT+ and one TEL+ or senescent cell line for comparison.

FIGS. 11A-11B illustrate certain aspects of the analysis of super-long telomeres in U2OS (green), SK-MEL-2 (red), Saos-2 (gray), UMUC3 (yellow), LNCaP (blue), and IMR90-S (purple), in accordance with some embodiments. For all cell lines, the colored bars represent the longest measured telomere from a specific chromosome arm. FIG. 11A: Super-long telomeres from all p chromosome arms that were detected by the SMTA-OM. FIG. 11B: Super-long telomeres from all q chromosome arms that were detected by the SMTA-OM. Chromosome arms that are not depicted did not have measurements from at least one ALT+ and one TEL+ or senescent cell line for comparison.

FIG. 12 illustrates certain aspects of the coefficient of variation (CV) for overall telomere length including end telomeres, TFEs, and ITS measurements, in accordance with some embodiments. The overall telomere mean lengths and standard deviations were calculated for all 6 cell lines. The CV was then calculated by dividing the standard deviation by the overall telomere mean length. The cell lines are grouped by telomere maintenance mechanism pathways. First is senescent IMR90 cells in purple, followed by two TEL+ cells, UMUC3 in yellow and LNCaP in blue, and lastly three ALT+ cells: U2OS in green, SK-MEL-2 in red, and Saos-2 in gray. The values adjacent to the ‘X’ located in the center of the box are the mean CVs.

FIGS. 13A-13F show some of the analysis results of cell lines with the SMTA-OM assay, in accordance with some embodiments. In FIGS. 13A-13F, mean telomere lengths, standard deviations, and percentages of molecules with TFE, ITS+, and ITS− for each analyzed chromosome arm were calculated for each cell line.

FIG. 14 shows the p values of the statistical analysis of the cell lines, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In the study described herein (“the present study”), it was discovered that ALT+ cancers have certain telomere signatures that distinguish them from the more common TEL+ cancers. These telomere signatures can be found by global analysis of telomere lengths, such as in one, some or all chromosomes. The present study further provides an assay (sometimes referred to as “single molecule telomere assay via optical mapping” or “SMTA-OM”) that is useful for the analysis of telomere lengths.

Accordingly, in some aspects, the present invention is directed to a method of categorizing cancers.

Furthermore, since ALT+ cancers are particularly susceptible to the inhibition of certain genes/proteins, such as FANCM, ATR, and PARP, the reliable categorization of the cancers further enables the treatment of these cancers.

Accordingly, in some aspects, the present invention is directed to a method of treating cancers.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

### Method of Categorizing Cancers

In some aspects, the present invention is directed to a method of categorizing a cancer. In some embodiments, the cancer is categorized to one of two types: (A) a cancer with activated expression of telomerase (TEL+), or (B) a cancer utilizing an alternative lengthening of telomere pathway (ALT+ cancer).

In some embodiments, the method comprises performing a global analysis of telomere lengths in cancer cells of the cancer. The method of performing global analysis of telomere lengths is described elsewhere herein, as well as in US Patent Publication No. 2018/0105867. The entirety of this reference is hereby incorporated herein by reference.

In some embodiments, an increased abundance of fusions/internal telomere-like sequence (ITS+) in the cancer cells relative to relative to a reference abundance indicates a cancer utilizing an alternative lengthening of telomere pathway (ALT+ cancer).

In some embodiments, an increased abundance of fusions/internal telomere-like sequence loss (ITS−) in the cancer cells relative to a reference abundance indicates an ALT+ cancer.

In some embodiments, an increased abundance of telomere-free ends (TFEs) in the cancer cells relative to a reference abundance indicates an ALT+ cancer.

In some embodiments, an increased abundance of super-long telomeres in the cancer cells relative to a reference abundance indicates an ALT+ cancer. In some embodiments, the term “super-long telomere” refers to telomere that is about 20% longer, about 30% longer, about 50% longer, about 75% longer, about 100% longer, about 150% longer, about 200% longer, about 300% longer, about 500% longer, about 750% longer, or about 1000% longer than telomere of the tissue/cell from which the cancer originates, or an average of various types of cancers.

In some embodiments, an increased level of heterogeneity of telomere lengths at the whole-genome level in the cancer cells relative to a reference level indicates an ALT+ cancer.

In some embodiments, the reference abundances/reference levels herein are based on abundances/levels of a healthy tissue/cell, such as a healthy tissue/cell from which the cancer originated. In some embodiments, the reference abundances/reference levels herein are based on averages of various types of cancers.

In some embodiments, performing the global analysis of telomere lengths in the cancer cells comprises: contacting genomic DNA of the cancer cells with a guide RNA (gRNA) having a portion complementary to a telomere-specific motif, and a nickase to introduce a nick in the telomere; contacting the nicked DNA with a polymerase and a nucleotide labeled with a first dye such that the nucleotide labeled by the first dye is incorporated into the nicked telomere; and detecting the telomere according to a signal of the first dye.

In some embodiments, the gRNA guides the nikase to the telomere, thereby allowing the nickase to introduce the nick in the telomere.

In some embodiments, the gRNA is complementary to a telomere repeat (TTAGGG)n.

In some embodiments, the nikase is a Cas9 nickase.

In some embodiments, the method further comprises labeling the genomic DNA of the cancer cells with a second dye labeling the genomic DNA; and detecting the genomic DNA according to a signal of the second dye. In some embodiments, the second dye labels the entirety of the genomic DNA regardless of the specific nucleotide sequences.

In some embodiments, the method further comprises generating a recognition pattern on each chromosome of the cancer cells; and matching sections of the cancer cell chromosomes with chromosome sections of a healthy cell according to the recognition pattern. In some embodiments, generating the recognition patterns allows mapping each section of the genomic DNA to the corresponding sequences in a healthy cell, such as those from which the cancer originates.

In some embodiments, generating the recognition pattern the chromosomes of the cancer cells comprises: contacting the genomic DNA with a motif-specific nicking endonuclease, thereby producing a second nick in the genomic DNA at the motif sequence; and contacting the nicked DNA with a polymerase and a nucleotide labeled with a third dye such that the nucleotide labeled with the third dye is incorporated into the nicked DNA at the motif sequence location.

In some embodiments, the motif-specific nicking endonuclease is Nt.BspQI. Since Nt.BspQI recognizes the DNA sequence GCTCTTCN, which are distributed in the genome in known patterns, the recognition pattern generated by labeling the Nt.BspQI nicking sites can be mapped to the known pattern to identify the genomic DNA molecules being processed.

In some embodiments, the third dye is the same as the first dye or the third dye produce a same signal as the first dye. In some embodiments, the third dye is different from the first dye or the third dye produce a different signal from that produced by the first dye.

In some embodiments, the first dye, the second dye, and/or the third dye are fluorescent dyes.

In some embodiments, detecting the telomere according to the signal of the first dye comprises analyzing the genomic DNA labeled with the first dye according to a nanochannel array method. In some embodiments, the nanochannel array methods further analyzes the signals of the second dye and/or the third dye. The nanochannel array analysis is described in, for example, Lam et al. (Nat. Biotechnol. 2012; 30:771-776).

### Method of Treating Cancer

In some aspects, the present invention is directed to a method of treating cancer in a subject in need thereof.

In some embodiments, the method comprises determining a cancer as an ALT+ cancer, such as according to the categorization method described herein, and administering to the subject an effective amount of an inhibitor for Fanconi anemia, complementation group M (FANCM), an inhibitor for ataxia telangiectasia and Rad3-related (ATR), or an inhibitor for poly (ADP-ribose) polymerase (PARP).

In some embodiments, the inhibitor for FANCM is at least one selected from the group consisting of MM2 peptide (described in Lu et al. Nat Commun. 2019; 10: 2252), and an RNA interference molecule targeting FANCM.

In some embodiments, the inhibitor for ATR is at least one selected from the group consisting of ART0380, ATG-018, ATRN-119, AZ20, berzosertib, Camonsertib (RP-3500), ceralasertib, CGK 733, dactolisib, elimusertib, ETP-46464, HAMNO (NSC-111847), IMP9064, SKLB-197, Schisandrin B, Torin 2, Tuvusertib, VE-821, VX-803 (M4344), and an RNA interference molecule targeting ATR.

In some embodiments, the inhibitor for PARP is at least one selected from the group consisting of 3-aminobenzamide, CEP 9722, E7016, iniparib, niraparib, olaparib, pamiparib, rucaparib, talazoparib, veliparib, and an RNA interference molecule targeting PARP.

### Downregulating FANCM, ATR or PARP by RNA Interference

In some embodiments, the FANCM, ATR or PARP is inhibited by a nucleic acid that downregulates the activity and/or expression level of these genes by the means of RNA interreference.

In some embodiments, the nucleic acid that inhibits FANCM, ATR or PARP by the means of RNA interreference includes an isolated nucleic acid. In other embodiments, the modulator is an RNAi molecule (such as but not limited to siRNA and/or shRNA and/or miRNAs) or antisense molecule, which inhibits FANCM, ATR or PARP expression and/or activity. In yet other embodiments, the nucleic acid comprises a promoter/regulatory sequence, such that the nucleic acid is preferably capable of directing expression of the nucleic acid. Thus, the instant specification provides expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of FANCM, ATR or PARP. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; Engelke, Ed., RNA Interference (RNAi) Nuts & Bolts of RNAi Technology, DNA Press, Eagleville, PA (2003); and Hannon, Ed., RNAi A Guide to Gene Silencing, Cold Spring Harbor Laboratory Press, Cold Spring Harbor, NY (2003). Soutschek et al. (2004, Nature 432:173-178) describes a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the instant specification also includes methods of decreasing levels of FANCM, ATR or PARP using RNAi technology.

In certain embodiments, the instant specification provides a vector comprising an siRNA or antisense polynucleotide. In other embodiments, the siRNA or antisense polynucleotide inhibits the expression of FANCM, ATR or PARP. The incorporation of a desired polynucleotide into a vector and the choice of vectors is well-known in the art.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In certain embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Following the generation of the siRNA polynucleotide, a skilled artisan will understand that the siRNA polynucleotide has certain characteristics that can be modified to improve the siRNA as a therapeutic compound. Therefore, in some embodiments, the siRNA polynucleotide is further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

In certain embodiments, an antisense nucleic acid sequence expressed by a plasmid vector is used to inhibit FANCM, ATR or PARP protein expression. The antisense expressing vector is used to transfect a mammalian cell or the mammal itself, thereby causing reduced endogenous expression of FANCM, ATR or PARP.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the instant specification may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the instant specification include oligonucleotide derivatives known in the art which have improved biological activity compared to unmodified oligonucleotides (see U.S. Pat. No. 5,023,243).

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1: High-Throughput Single-Molecule Telomere Characterization

In Example 1, a novel method that enables global subtelomere and haplotype-resolved analysis of telomere lengths at the single-molecule level was developed. An in vitro CRISPR/Cas9 RNA-directed nickase system directs the specific labeling of human (TTAGGG)n DNA tracts in genomes that have also been barcoded using a separate nickase enzyme that recognizes a 7-bp motif genome-wide. High-throughput imaging and analysis of large DNA single molecules from genomes labeled in this fashion using a nanochannel array system permits mapping through subtelomere repeat element (SRE) regions to unique chromosomal DNA while simultaneously measuring the (TTAGGG)n tract length at the end of each large telomere-terminal DNA segment. The methodology also permits subtelomere and haplotype-resolved analyses of SRE organization and variation, providing a window into the population dynamics and potential functions of these complex and structurally variant telomere-adjacent DNA regions. At its current stage of development, the assay can be used to identify and characterize telomere length distributions of 30-35 discrete telomeres simultaneously and accurately. The assay's utility is demonstrated using early versus late passage and senescent human diploid fibroblasts, documenting the anticipated telomere attrition on a global telomere-by-telomere basis as well as identifying subtelomere-specific biases for critically short telomeres. Similarly, the present study present the first global single-telomere-resolved analyses of two cancer cell lines.

### Example 1-1

In humans, telomeres are nucleoprotein complexes made up of tandem 5′TTAGGG3′ DNA repeats and associated proteins located at the ends of all 46 chromosomes. Telomere (TTAGGG)n tract loss beyond a certain threshold changes the telomere structure (“uncapping”), which causes telomere dysfunction and leads to senescence or apoptosis. The senescence threshold is cell line specific for human diploid fibroblasts (HDFs), with the shortest single-telomere length detected at senescence 300 bp. In vivo, aberrant senescence or apoptosis can disrupt tissue microenvironments and contribute to aging and cancer. When tumor suppressor pathways are compromised in the presence of dysfunctional telomeres, further telomere attrition occurs and leads to telomere fusions; genome instability ensues and contributes to the formation of a tumor.

Currently, there are several methods to measure the length of telomere repeats each with their own advantages and disadvantages. Terminal restriction fragment (TRF) estimates the average telomere length of a population of cells with a resolution of 1 kb. TRF requires at least 1.5 μg of DNA, can overestimate the telomere length by several kilobases, and is not sensitive to very short telomeres. The quantitative PCR (qPCR) method was developed to decrease the amount of DNA required, with only 25-50 ng needed to estimate telomere repeat content of a sample. However, the method actually measures the ratio of total telomere repeat content in a sample relative to a single-copy gene (T/S ratio), which in turn must be related to base pair of telomere using a standard curve of samples whose telomere lengths have been determined by another method (typically TRF analysis), thus providing estimates of average telomere length per sample. Single-telomere length analysis (STELA) and quantitative fluorescence in situ hybridization (Q-FISH) were developed to detect and measure the length of specific telomeres. STELA can measure single-telomere lengths with a resolution of 0.1 kb and sometimes identifies allelic differences in an individual telomere length. However, it requires unique priming sites in subtelomere regions that are enriched in duplications and is usually limited to subtelomeres XpYp, 2p, 11q, 12q, and 17p. Q-FISH of telomere repeats (Q-FISH) requires only 15-20 metaphase cells per sample and measures telomeres on all individual chromosome arms. This method is also able to identify chromosome ends without detectable repeats (<0.5 kb), as well as chromosome fusion occurrences. A major disadvantage of Q-FISH is that it is limited in the analysis of cells currently in metaphase and is unable to measure telomeres in terminally senescent cells or cells that are no longer able to divide. With the current methods to measure telomere length, it is impossible to efficiently acquire global subtelomere and haplotype-resolved telomere length data.

Normal human telomere lengths vary between chromosomes within the same cell and even homologous chromosome arms. The mechanisms regulating subtelomere-specific and haplotype-specific telomere lengths in humans are understudied and poorly understood, primarily because of technical limitations in obtaining these data globally. Although the relative telomere lengths of single chromosome arms within individuals appear to vary considerably with genetic background (including specific subtelomere haplotypes), there have been some chromosome arm-specific telomere length trends documented. For example, telomeres on chromosome arms 17p, 19p, and 20q have been identified as some of the shortest, whereas 1p, 3p, 2p, and 4q contain telomeres among the longest.

Studies measuring average telomere lengths have established a relationship between shorter average telomere lengths and increasing age, age-related diseases, and mortality. However, there have been numerous studies suggesting that the shortest individual telomeres in cells mediate the biological effects associated with short average telomere lengths via telomere dysfunction; the shortest telomere or a small number of short telomeres in cells rather than the average telomere length is critical for chromosome stability and cell viability. The identity and frequency of specific critically short telomeres may thus be the most useful telomere biomarker for aging and age-related diseases, including cancer. Gaining a better understanding of telomere length regulation in healthy humans, including factors involved in single telomere-specific regulation of (TTAGGG)n tract length and stability, may lead to more precise telomere biomarkers and better approaches to defining the role of telomeres in diseases. However, with the current methods to measure telomere length, it is impossible to acquire such global haplotype-resolved telomere length data.

To address this need, the present study developed a method that simultaneously measures individual telomere (TTAGGG)n tract lengths and identifies their physically linked subtelomeric DNA. This assay can also be used to identify chromosome ends lacking detectable telomere sequences, characterize novel subtelomeric structural variants and haplotypes, and discover previously uncharacterized subtelomere regions of human genomes. Here, this technology and its application to the analysis of single-telomere lengths in two cancer cell lines and to single-telomere length changes in the IMR90 cellular aging model are described.

### Example 1-2: Results

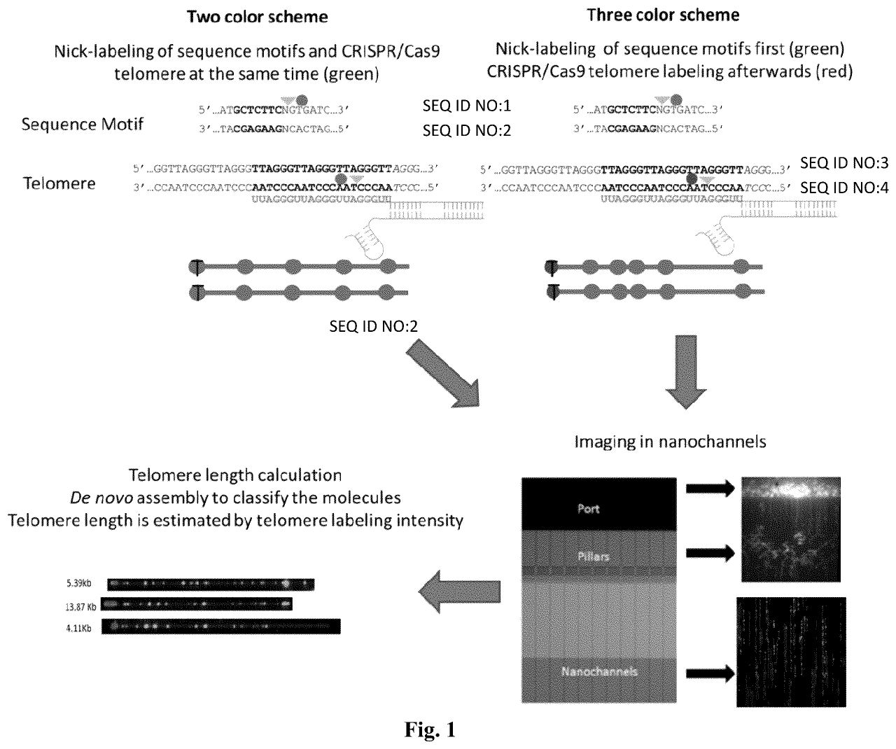

FIG. 1 shows an overview of the single-molecule telomere length assay. For the telomere/subtelomere colabeling strategy to work, the genome-wide sequence motif-dependent nick-labeling (Jo et al., Proc Nal Acad Sci 104: 2673-2678; Xiao et al., Nucleic Acids Res 35: e16; Das et al., Nucleic Acids Res 38: e177) must differentiate individual subtelomeres, and the telomere labeling must be specific and proportional to (TTAGGG)n tract lengths. Nt.BspQI, which has been used widely to nick-label (GCTCTTC) sites genome-wide for large single-molecule-based whole-genome map assemblies, was selected initially for sequence motif mapping (FIG. 1; Mak et al., Genetics 202: 351-362). For telomere-specific labeling, the present study developed a CRISPR/Cas9 nickase (Cas9n) (Ran et al., Nat Protoc 8: 2281-2308)-based protocol directed to telomere (TTAGGG)n tracts by a synthetic guide RNA containing the sequence UUAGGGUUAGGGUUAGGGUU (SEQ ID NO:5), which is complementary to the 3′ to 5′ telomere DNA repeat strand and followed by the protospacer adjacent motif (PAM) NGG. Optimal telomere labeling conditions were defined empirically, with the conditions found to provide maximal overall telomere fluorescent intensity used for all subsequent experiments. The present study developed two-color and three-color labeling schemes for the single-molecule telomere length assay (FIG. 1). In both schemes, the same gRNA is used to nick-label the telomeric repeats, while the subtelomeric regions are globally nick-labeled using Nt.BspQI to target the GCTCTTC motif. In the two-color scheme, the Cas9n and Nt.BspQI are incubated with the human genomic DNA and simultaneously nick their respective regions. All nicks are labeled with green fluorescently labeled nucleotides using Taq DNA polymerase. In the three-color scheme, Nt.BspQI is first incubated with the human genomic DNA and nicks the GCTCTTC motif. These nicks are labeled with green fluorescently labeled nucleotides using Taq DNA polymerase. The unincorporated green fluorescent nucleotides are removed with shrimp alkaline phosphatase (SAP). The labeled DNA molecules are then nicked with Cas9n and labeled with red fluorescently labeled nucleotides using Taq DNA polymerase. The labeled DNA molecules are then optically imaged in a high-throughput manner using nanochannel arrays (Lam et al., Nat Biotechnol 30: 771-776). De novo assembly is performed using the unique subtelomeric Nt.BspQI patterns, and the assemblies are then mapped to subtelomeric reference sequences (human hg38). The individual telomere-terminal DNA molecules thus assembled are used for calculating the telomere length. This allows for the simultaneous identification of the chromosome arms and quantitation of the linked single-telomere lengths.

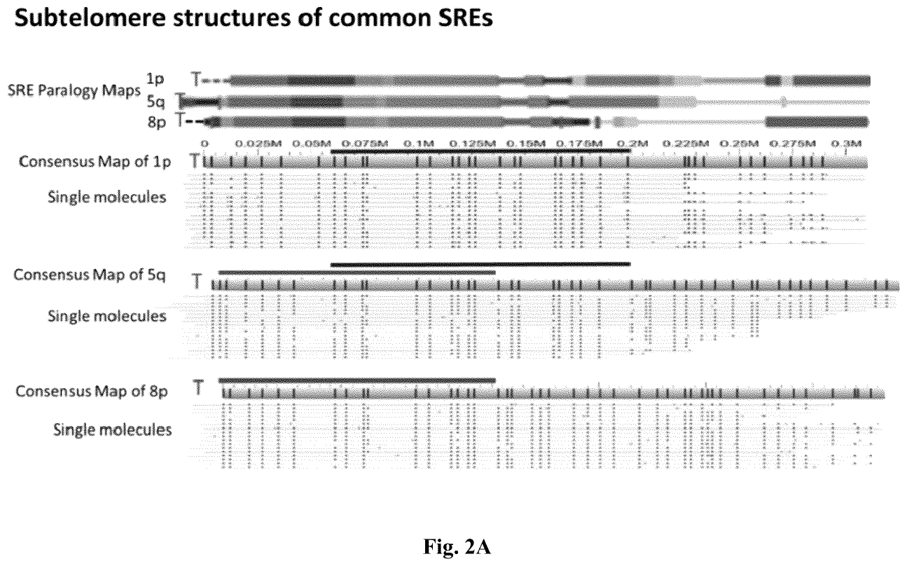

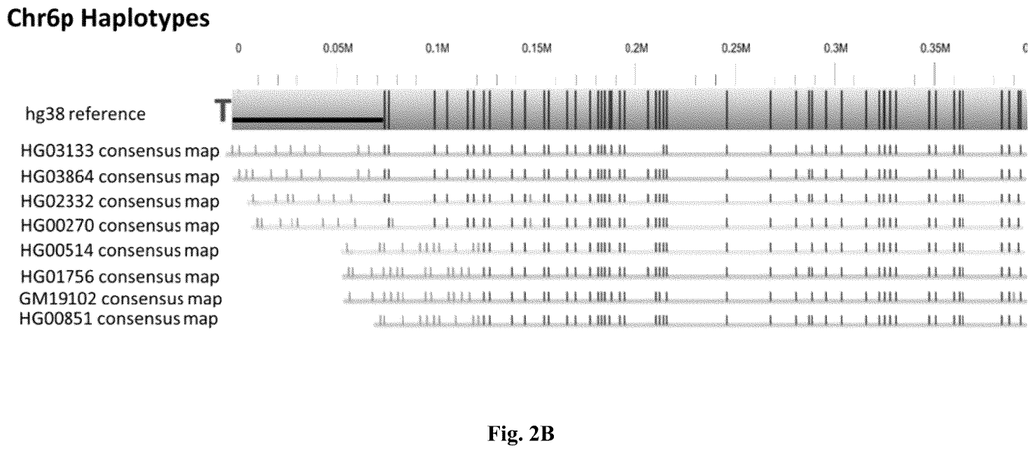

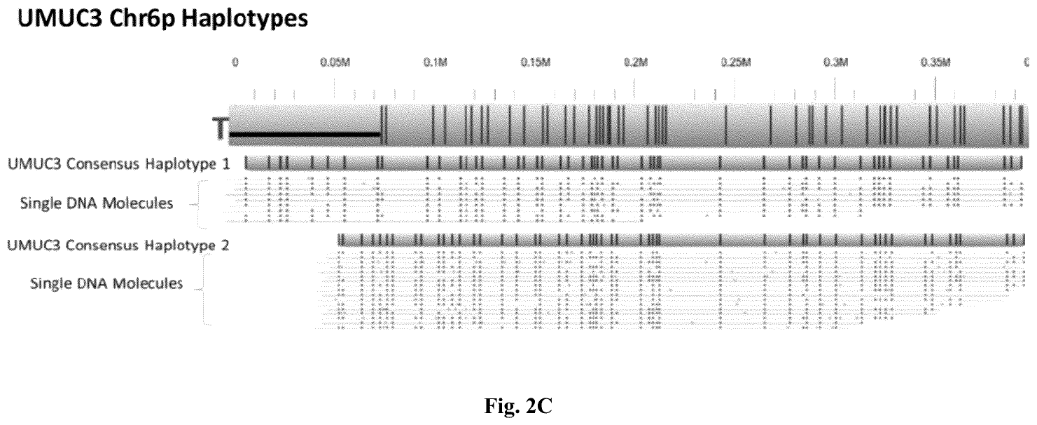

Subtelomeric repeat element (SRE) regions positioned immediately adjacent to telomere (TTAGGG)n tracts have complicated efforts to distinguish and identify single chromosome ends in human genomes, effectively precluding global single-telomere analysis at the molecular level. These regions contain highly similar (90%-99.9% sequence identity) segmental duplications, large structural variations, and reference sequence gaps and misassemblies. While most SRE regions are 40-150 kb in size, long SRE regions of up to ˜300 kb have been identified in a few subtelomeres, including 1p, 8p, and 5q. Motif-dependent nick-labeling of large genomic DNA molecules followed by assembly of mapped single large DNA molecules can effectively distinguish SRE regions and permit connection of chromosome ends harboring the largest SRE regions with specific subtelomeres. FIG. 2A demonstrates this for the 1p, 5q, and 8p subtelomeric regions of the HG11381 genome. These subtelomeric regions share some SREs but differ in others, as shown in the paralogy maps of these subtelomere reference sequences (FIG. 2A, top, “paralogy map”). Following motif-dependent nick-labeling of the HG11381 genome, the 1p, 5q, and 8p subtelomeres were de novo assembled into unique consensus maps from the mixture of single long DNA molecules (FIG. 2A, bottom). 1p and 5q share similar duplicons up to 200 kb from the telomere according to the paralogy map, which is supported in part by the same sequence motif patterns from 55-200 kb (blue bar). However, the sequence motif patterns are clearly distinguishable between 1p and 5q from 0-55 kb, even though this region contains several similar duplicons. As indicated with the red bars in FIG. 2A consensus maps, 5q and 8p share very similar duplication structure and organization until 130 kb from the telomere, where the sequence motif patterns start to differ. Based on the above differences, only a 150-kb region is needed to differentiate these three highly similar subtelomeric regions. This is true for all the other subtelomeric chromosome arms sharing similar SREs; furthermore, definitive localization to subtelomere-specific one-copy DNA (gray line segments in the paralogy maps) is typically achieved using large DNA molecules in this size range. This genome mapping method with long DNA molecules provides a unique tool to track and identify individual SRE variants within families of SREs and to connect each chromosome end with a specific subtelomere.

The Nt.BspQI nickase nick-labeling method can also identify structurally variant haplotypes in highly variable subtelomeric regions. Taking advantage of a related project constructing hundreds of human genome sequence motif maps with Nt.BspQI nick-labeling (GCTCTTC), the present study analyzed and compared assembled subtelomeric regions over 100 genomes to determine whether the Nt.BspQI nick-labeling can distinguish subtelomeric identities and structural variant haplotypes. FIG. 2B shows eight major variant haplotypes of Chromosome 6p detected in this large data set. The consensus maps each contain labels that do not align to the hg38 reference and differ from one another (light blue lines). FIG. 2C identifies two distinct 6p haplotypes found in the cancer genome of UMUC3.

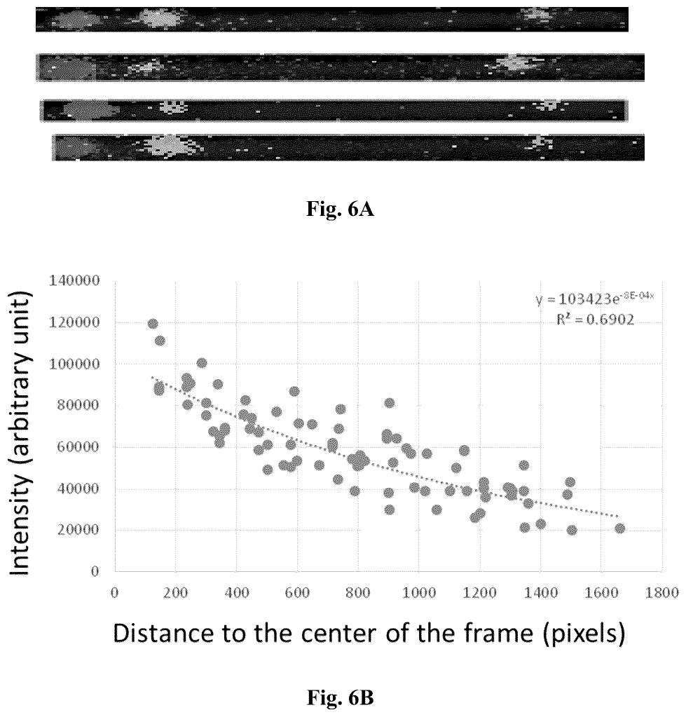

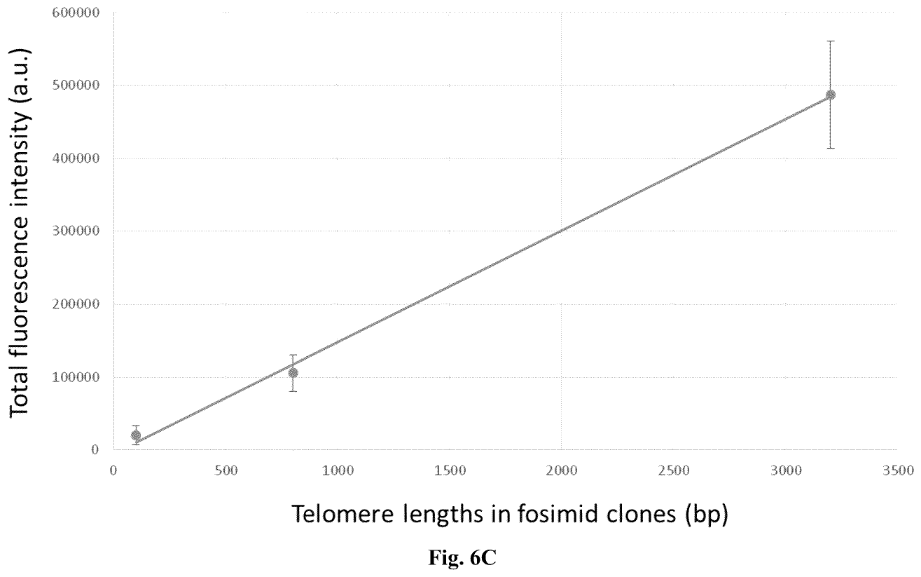

After confirming that global sequence motif labeling can differentiate subtelomeres, the present study optimized the CRISPR/Cas9 telomeric labeling using a circular fosmid containing an 800-bp tract of telomeric repeats cloned from the human chromosome arm 8q. There was no fluorescent labeling of the telomeric repeats (TTAGGG) without the presence of either Cas9n or gRNA. The fluorescent labeling of telomeric repeats was detected only when both Cas9n and gRNA were present in the reaction (data not shown). The present study then combined the Cas9n (TTAGGG) and Nt.BspQI (GCTCTTC) to nick-label the telomere and the adjacent subtelomeric DNA of the fosmid at the same time. After linearization of fosmid DNA with the enzyme NotI, the Nt.BspQI labeling pattern of the fosmid matched the reference sequence, and the telomeric labeling was always at the end of the molecules (FIG. 6A), indicating the telomeric labeling.

There are two possible methods to measure the telomere repeat lengths from fluorescent labels: based on the contour of telomeric labeling and the intensity of telomeric labeling. The longer the telomere, the more pixels it will occupy. However, the ends of DNA molecules tend to fold back onto themselves which affects the length measurements. More importantly, even with a single-point emitter, several pixels collect photons due to photon scattering. By use of the contour method, it is impossible to differentiate and resolve a telomere length<1 kb as all telomeres<1 kb will occupy the same number of pixels. The intensity method uses the total intensity of the telomeric labeling. It was reasoned, the longer the telomere, the more frequently Cas9n will nick the (TTAGGG)n tract and fluorescent nucleotides become incorporated. The total intensity should therefore be proportional to the telomere length. The present study used fosmids with known telomere length to quantify the system and normalize the intensity measurement for estimating single-telomere lengths (FIGS. 6A-6C), and then extrapolated the intensities/base pair (TTAGGG)n tract from these standards to estimate base pair in genomic (TTAGGG)n tracts on single molecules based upon the measured fluorescent intensities.

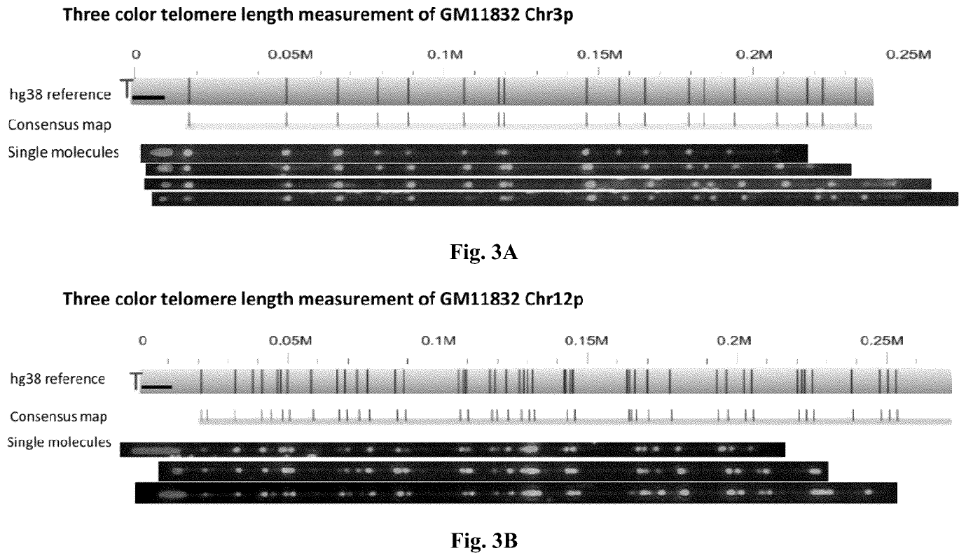

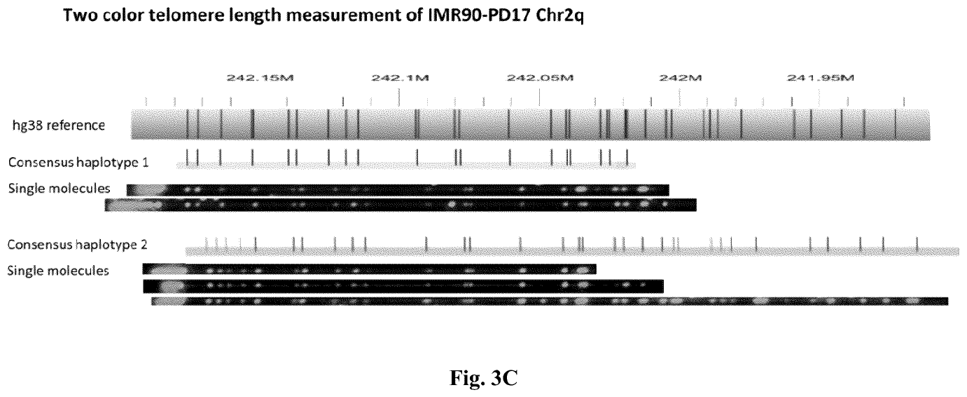

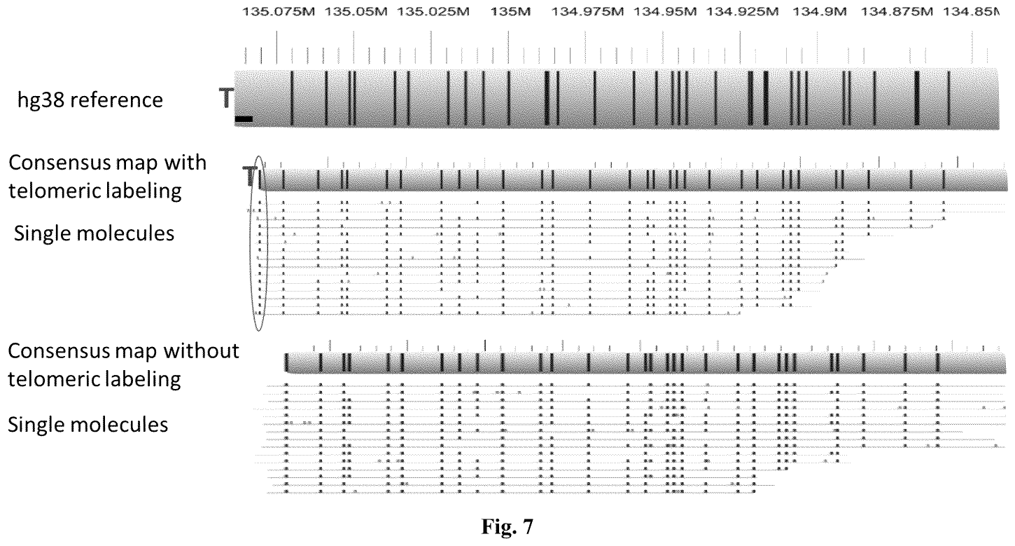

The initial results of the two colabeling strategies for the single-molecule telomere length assay are shown in FIGS. 3A-3C. In the first application, the present study tested the three-color scheme on human genomic DNA sample GM11832. The results for Chromosomes 3p and 12p are shown in FIGS. 3A and 3B, respectively. The telomeres are clearly labeled in red at the end of each molecule. The three-color scheme easily differentiates subtelomeres and labels genomic (TTAGGG)n tracts at the ends of the mapped molecules but is a time-consuming assay that involves two separate nick-labeling reactions with an intermediate step to remove the free fluorescently labeled nucleotides. This additional step along with frequent pipetting tend to break the DNA into smaller fragments, resulting in fewer long DNA fragments ideal for distinguishing the larger SRE regions. The present study hypothesized that the two-color scheme could also differentiate subtelomeres and label (TTAGGG)n tracts at the ends of long mapped subtelomere fragments while at the same time maintaining longer DNA molecules. This scheme was first tested with the IMR90 cell line, and indeed, this was the case. The single molecules produced from both color schemes generated the same consensus maps that were used to identify single telomeres and haplotypes, as well as labeled (TTAGGG)n tracts at the ends of the mapped molecules. The consensus maps of both haplotypes as well as examples of single molecules for Chromosome 2q from IMR90-PD17 are shown in FIG. 3C. The ends of the molecules were labeled in green. As expected, when Cas9 was not used on the same sample, the ends of the molecules with the sample pattern were not labeled (FIG. 7). The two-color scheme generates longer DNA molecules for mapping, has a simpler experimental procedure, and involves much less imaging time with two-thirds of the data storage requirements.

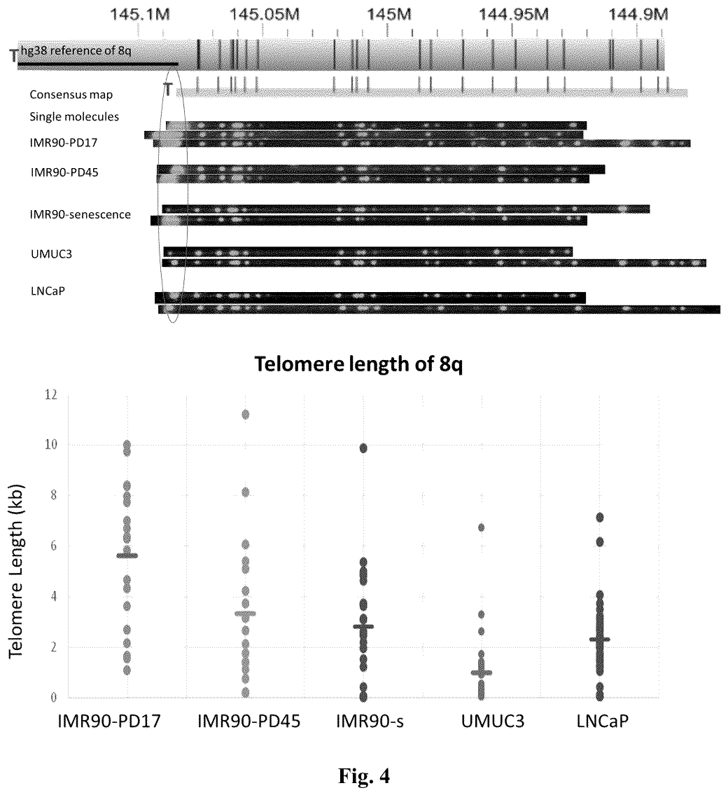

Next, the present study applied the two-color labeling scheme to identify individual telomere lengths in the IMR90 fibroblast aging model cell line at early passage, late passage, and senescence, as well as the UMUC3 bladder cancer and the LNCaP prostate cancer cell lines. Typical raw imaging results used for determination of the average single-telomere lengths, in this case for Chromosome 8q, are shown at the top of FIG. 4. The consensus maps aligned well to the hg38 human reference. It is visually obvious by the intense end telomere labels that IMR90-PD17 has the relatively longest telomeres. At population doubling of 45, the telomeres get shorter. Interestingly, when the IMR90 cells were senescent, a fraction of the 8q telomeres were very short, whereas others were still relatively long. The individual single-molecule telomere length distribution for 8q is plotted for each cell culture sample at the bottom of FIG. 4B, lower panel, where each spot is a single-telomere length measurement and the mean size is indicated by the horizontal bar for each sample. The broad size ranges of single telomere measurements in a given sample is similar to what is found for the PCR-based STELA single-telomere length distributions for bulk cell cultures. These reflect in part the mixed clonal subpopulations of IMR90 cells doubling at different rates within the average cell division rate designated by the population doubling number of the cell culture overall, along with mixed clone-specific telomere attrition rates affected by human telomere rapid deletion (TRD) events and telomere end-processing, among other potential factors. The relatively large standard deviation associated with each average single-telomere length in the IMR90 samples thus reflects primarily single cell-dependent biological variation rather than measurement error; coefficients of variation (CVs) calculated for average single-telomere lengths in technical replicates using DNA from the IMR90-PD17 cell culture (inter-assay, identical samples processed and analyzed weeks to months apart) were 14.6%, which is in the ballpark of CVs reported for published inter-assay telomere measurements using Q-FISH, Flow-FISH, and qPCR.

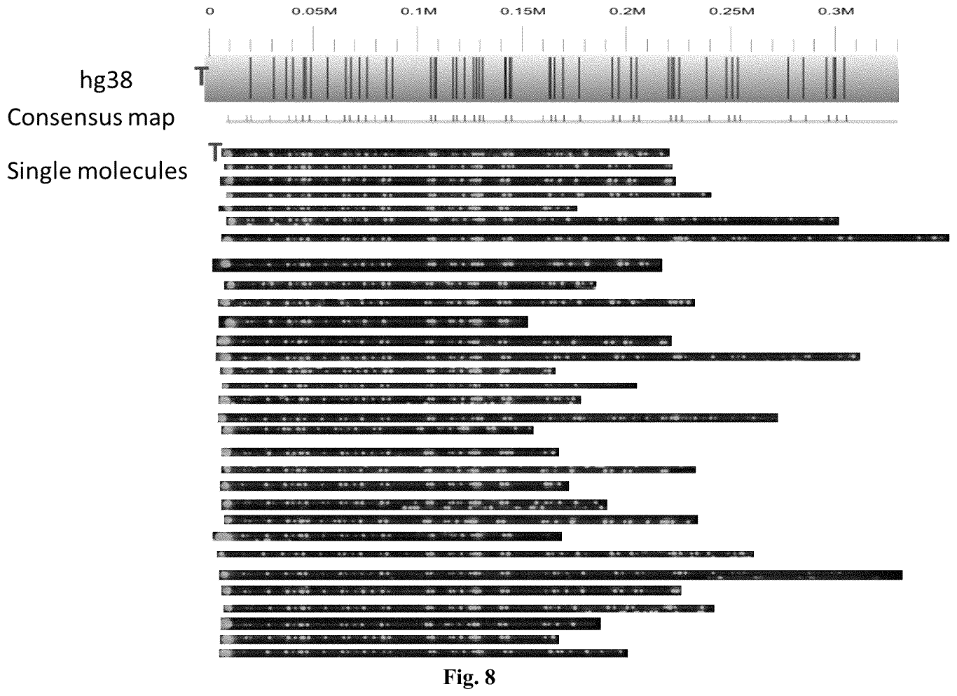

Globally, the present study could measure 36 out of 46 telomeres with about 30 molecules per telomere and detect telomere tracts<100 bp. The present study did not observe any mapped, apparently terminal, large DNA molecules totally lacking telomere signal, which may indicate that “telomere-free ends” documented by Q-FISH studies of metaphase chromosomes may in fact retain extremely short telomeres that are below the detection limit for Q-FISH but are still detectable using the technique. A full set of raw single-molecule images are shown in FIG. 8. The chromosome arms 13p, 14p, 15p, 21p, and 22p could not be identified and measured due to the lack of reference sequences for acrocentric short-arm telomeres. The telomeres of XpYp were also not measured because there were no contigs that spanned the telomere adjacent sequences of the XpYp subtelomere reference, possibly due to many gaps in the hg38 reference and a large highly variable region containing minisatellite repeats near this telomere. Lastly, 16p, 17p, 19q, and 22q failed the assembly with most samples because of inverted nick pair (INP) sites. These are where two closely spaced nicking enzymes sites are found on opposite strands, which causes double-strand breaks in molecules to be mapped, that preclude the assembly on long continuous contigs.

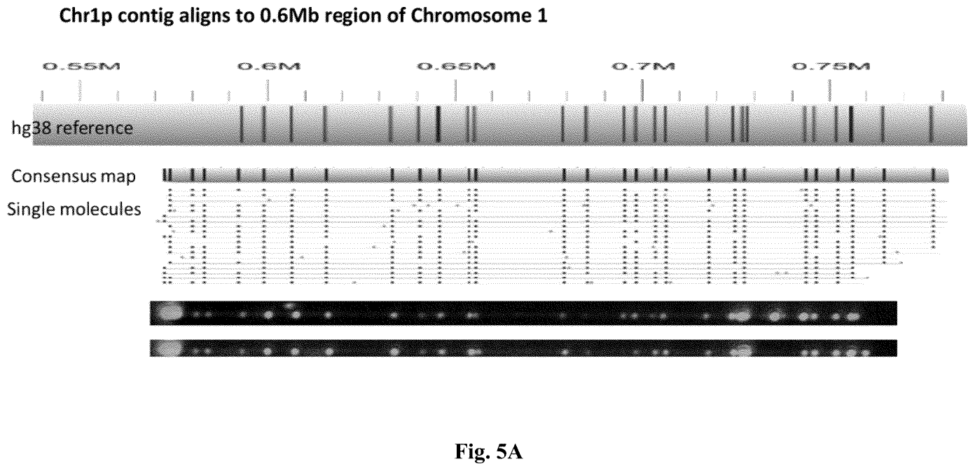

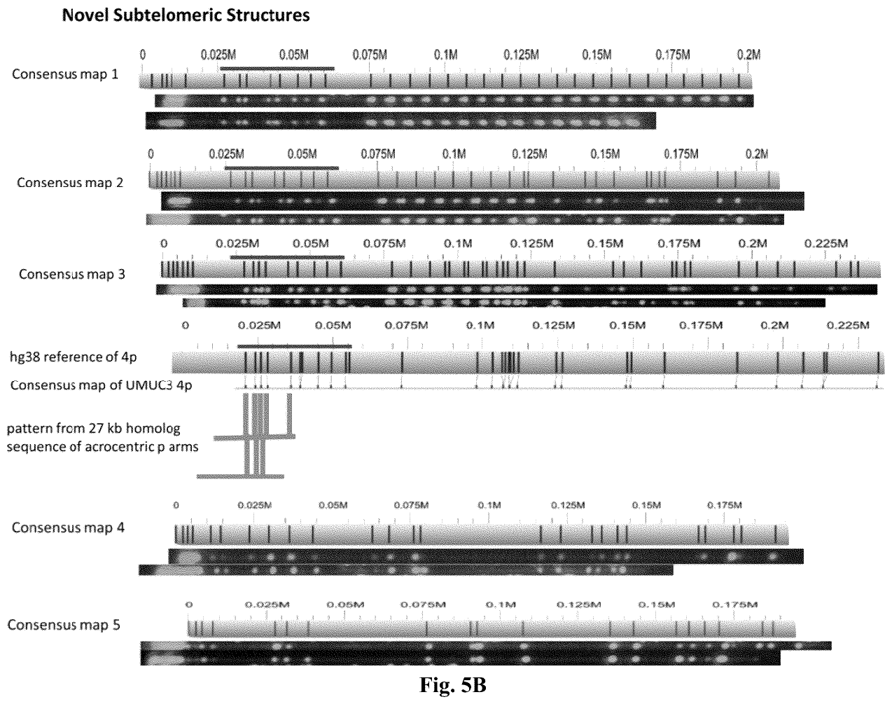

In addition to its utility for quantifying single-telomere lengths on large molecules linked to specific known telomeres, the specific fluorescent tagging of telomeres is a very powerful tool to identify previously uncharacterized subtelomeres. This makes it particularly valuable for improving the quality of subtelomeric reference sequences and for identifying new structurally variant subtelomeres. For example, the subtelomeric region 0-500 kb of Chromosome 1p arm is missing in all of the genomes mapped. However, the present study noticed that a 1p DNA-containing contig mapping 600 kb from the 1p telomere in hg38 contained either intense telomere gRNA-directed CRISPR/Cas9n-dependent labeling green end labels (two-color labeling scheme) or intense red labels (three-color labeling scheme) in all of the genomes analyzed (FIG. 5A). It was concluded that the experiments had identified the true 1p telomere and that 0-600 kb of hg38 is incorrect and should be removed from the reference assembly.

In addition, five consensus maps that could not be mapped to the hg38 reference were found to have a telomere label at one end (FIG. 5B). Consensus maps 1, 2, and 3 share a 40-kb conserved pattern, marked as the red bars in FIG. 5B. This pattern is similar to the pattern of Chromosome 4p of hg38 from 25 kb to 65 kb and corresponds to a segmental duplication that is shared by the subtelomeres of 4p, 4q, and many acrocentric short-arm subtelomeres. Taken together with the telomeric labels at the end of DNA molecules, it is strongly suggested these three consensus maps belong to the five unknown acrocentric chromosome ends 13p, 14p, 15p, 21p, and 22p. Interestingly, they all have 7-kb repetitive sequence units following this 40-kb conserved pattern. Consensus map 1 has the longest 7-kb repeat region (>140 kb), which makes the sequence assembly impossible with current sequencing technologies. Based on these patterns, the present study was able to locate more unaligned consensus maps with the same patterns from other genomes, with some>700 kb long. The other two unaligned consensus maps with telomeric labels (FIG. 5B, bottom) reflect yet to be identified subtelomere alleles that cannot be incorporated into the current reference sequence on the basis of their nickase mapping patterns.

### Example 1-3

Here, the present study developed novel methods that facilitate global subtelomere-specific analysis of human lengths at the single-molecule level. One of the key technology advances is to use the CRISPR/Cas9 genome editing system to covalently tag the telomeric repeats with fluorescent dyes. There have been reports using an EGFP-linked, deactivated dCas9 protein to label telomeres for imaging (Chen et al. 2013). While useful for some purposes (e.g., in vivo imaging), this approach is not appropriate for the application using nanochannel analysis for two reasons. First, the EGFP-dCas9 is noncovalently linked to the telomere target sequence, and some fraction will not remain bound during the nanochannel array analysis procedure, making accurate quantitation difficult. The method covalently incorporates fluorescently labeled nucleotides into the target telomere sequence and is therefore a far more stable interaction than the EGFP-dCas9. Second, the EGFP-dCas9 would introduce DNA-bound protein to the final sample, which would make it more difficult to load the sample into the nanochannels.

For the first time, large numbers of individual telomere lengths can be tracked efficiently in the context of total genomic DNA, as well as cis-factors influencing their length regulation and stabilities potentially evaluated. This new experimental capability may lead to much more complete and powerful analyses of genetic and epigenetic factors influencing telomere elongation, attrition, processing, and stability than those previously feasible using average telomere lengths. The methodology also connects subtelomere and haplotype specificity with individual SRE organization and variation, providing a window into the dynamics and potential functions of these complex and structurally variant telomere-adjacent DNA regions in telomere regulation and genome biology.

### Example 1-4: Methods

### Cell Preparation

UMUC3 cells, a human urinary bladder carcinoma cell line, were obtained from ATCC and cultured in Eagle's minimum essential medium (EMEM) containing Earle's salts, NEAA, and L-glutamine (2 mM; ATCC) with 10% fetal bovine serum (FBS; Corning). LNCaP cells, a human prostate cancer cell line, were obtained from ATCC and cultured in RPMI 1640 media containing 2 mM L-glutamine, 10 mM HEPES, 1 mM sodium pyruvate, 4500 mg/L glucose, and 1500 mg/L sodium bicarbonate (ATCC) supplemented with 10% FBS (Corning). IMR-90 cells, a human fetal lung fibroblast cell line, were obtained from Coriell Cell Repository and maintained in EMEM containing Earle's salts, NEAA, and L-glutamine (2 mM; ATCC) supplemented with 15% FBS (Corning). UMUC3 and LNCaP cells were passaged using 0.25% trypsin-EDTA (Gibco), and IMR-90 cells were passaged using 0.05% Trypsin-EDTA (Gibco).

### High-Molecular-Weight DNA Extraction

Mammalian cells were embedded in gel plugs, and high-molecular-weight DNA was purified as described in a commercial large DNA purification kit (BioRad no. 170-3592). Plugs were incubated with lysis buffer and proteinase K for 4 h at 50° C. The plugs were washed and then solubilized with GELase (Epicentre). The purified DNA was subjected to 2.5 h of drop-dialysis. It was quantified using Quant-iTdsDNA assay kit (Life Technology), and the quality was assessed using pulsed-field gel electrophoresis.

### Guide RNA Preparation

The seed sequence of 20 nucleotides complementary to the 3′-5′ strand of the telomere (UUAGGGUUAGGGUUAGGGUU, SEQ ID NO:5) was designed via a gRNA design tool (Feng Laboratory CRISPR design web tool at http://crispr.mit.edu). This seed sequence was incorporated into the crRNA. The crRNA and the universal tracrRNA were synthesized by GE Dharmacon. The telomere gRNA was created by preincubating the tracrRNA (0.1 nmol) and crRNA (0.1 nmol) on ice for 30 min.

### The Two-Color Labeling Scheme

The gRNA (2.5 μM) was incubated with 200 ng of Cas9 D10A (LabOmics), 1× NEBuffer 3 (New England BioLabs, NEB), and 1× BSA (NEB) at 37° C. for 15 min. The DNA (300 ng) and 5 U of Nt.BspQI (NEB) were added to the mixture and incubated at 37° C. for 60 min. The nicked DNA was labeled with 5 U of Taq DNA Polymerase (NEB), 1× green labeling mix (BioNano Genomics), and 1× Thermopol buffer (NEB) at 72° C. for 60 min. The nicks were repaired with 20 kU of Taq DNA Ligase (NEB), 1 mM NAD+(NEB), 100 nM dNTPs, and 1× Thermopol buffer (NEB) at 37° C. for 30 min. The small quantity (300 ng) of labeled genomic DNA required for a typical experiment is sufficient to generate at least 60× coverage of a genome, making it feasible to apply this method to small clinical sample sources such as blood.

### The Three-Color Labeling Scheme

The DNA (300 ng) was first nicked with 5 U of Nt.BspQI (NEB) in 1× NEBuffer 3 (NEB) at 37° C. for 2 h. The nicked DNA was then labeled with 5 U of DNA Taq Polymerase (NEB), 100 nM ATT0532-dUTP dAGC, and 1× NEBuffer 3.1 (NEB) at 72° C. for 60 min. The sample was treated with 0.3 U of SAP (USB Products) at 37° C. for 10 min and then 65° C. for 5 min. The gRNA (2.5 μM) was incubated with 200 ng of Cas9 D10A (LabOmics), 1× NEBuffer 3 (NEB), and 1× BSA (NEB) at 37° C. for 15 min. The green-labeled sample was then added to the reaction and incubated at 37° C. for 1 h. The Cas9 D10A nicks were labeled with 2.5 U of Taq DNA Polymerase (NEB), 1× IrysPrep labeling mix red (BioNano Genomics), and 1× NEBuffer 3.1 (NEB) at 72° C. for 60 min. The nicks were repaired with 20 kU of Taq DNA ligase (NEB), 1 mM NAD+(NEB), 100 nM dNTPs, and 1× NEBuffer 3.1 (NEB) at 37° C. for 30 min.

### DNA Loading and Imaging

After nick-labeling with either the two- or three-color schemes, the samples were treated with 6 mAU of QIAGEN Protease at 56° C. for 30 min, and the reaction was stopped with 1 μL of IrysPrep stop solution (BioNano Genomics). The DNA backbone was stained with 333 nM YOYO-1 (Invitrogen) and is shown in blue in all figures. The stained samples were loaded and imaged inside the nanochannels following the protocol described in Lam et al. (Nat Biotechnol 30: 771-776). BioNano Genomics labeling kit and IrysChip were used to generate the nick labeling data. The next generation mapping system from BioNano has dramatically improved the throughput; the custom-made systems are very similar to this new BioNano Genomics system. Each IrysChip contains two nanochannel devices, which can generate at least 60 Gb of data (molecules>150 kb). Normally, 60× coverage (180 Gb) is needed to generate 30 molecules of each chromosome end containing the telomeres. This assay runs 3 d, collecting over 24,000 images. It currently costs $1000 per sample to run whole-genome mapping. The image analysis was done using BioNano commercial software for segmenting and detecting DNA backbone based on the YOYO-1 staining similar to optical mapping method described in Lin et al. (Science 285: 1558-1562), and localizing the green labels by fitting the point-spread functions.

### De Novo Genome Map Assembly

Single-molecule maps were assembled de novo into consensus maps using software tools developed at BioNano Genomics, specifically Refaligner and Assembler (Mak et al., Genetics 202: 351-362). Briefly, the assembler is a custom implementation of the overlap-layout-consensus paradigm with a maximum likelihood model. An overlap graph was generated based on pairwise comparison of all molecules as input. Redundant and spurious edges were removed. The assembler outputs the longest path in the graph, and consensus maps were derived. Consensus maps are further refined by mapping single-molecule maps to the consensus maps, and label positions are recalculated. Refined consensus maps are extended by mapping single molecules to the ends of the consensus and calculating label positions beyond the initial maps. After merging of overlapping maps, a final set of consensus maps was output and used for subsequent analysis.

### Telomere Length Analysis

The molecules from the consensus maps, which were mapped to the ends of the individual chromosomes (hg38 reference) were designated as the molecules containing telomere and analyzed. These molecules contain additional labels not found in the reference, which were classified as telomere labels. The integrated fluorescence intensity of these labels was calculated after subtracting the background intensity. The intensity was then converted to base pairs based on standard established using fosmids (FIGS. 6A-6C).

### The Telomere Length Measurement Based on Labeling Intensity.

In the optical setup, the 532 nm laser was expended 28.3 times to cover the full CMOS camera. This results in the exponential decay of the laser power from the centroid. The present study used fosmids with known telomere length to quantify the system and normalize the telomere length measurements.

FIG. 6A shows typical fosmid molecules with 800 bp telomeres, green dots representing Nt.BspQI sequence motifs, and red dots for CRISPR/Cas9 telomeric labeling. The DNA backbone is blue. 99.3% of the fosmid molecules ware labeled at telomeric sites. The total raw intensity of telomeric labeling from individual fosmids is plotted in FIG. 6B. The telomere labeling intensity is following the laser power exponential decay. The intensity is the highest close to the center of the imaging frame, and the weakest at the edges. After normalizing the telomeric intensity to the center of the imaging frame according to the laser power exponential decay. Use the center of the intensity to represent the true 800 bp telomeric repeats, the average telomere length is calculated to be 814 bp±195 bp. The present study also tested the system with another fosmid of 100 bp telomeric repeats. 93.4% of fosmids are labeled at telomeric sites, the average telomere length is calculated to be 153 bp±97 bp. The shorter telomere length measurements show higher variation. Clearly, the method herein allows the detection of telomeric repeats as short as 100 bp. To make the calibration more accurate, it is necessary to have a standard of a few kb. However, it is impossible to stably clone several kb of telomere sequence. The present study decided to randomly select 4 molecules of the fosmids containing 800 bp telomere as a group to simulate a 3.2 kb telomere. The average telomere length is calculated to be 3056 bp±456 bp, which is in good agreement with expected results. The calibration is summarized in FIG. 6C.

### Comparison Between Cas9 Labeling and No Cas9 Labeling

For the IMR90 and UMUC cell lines, the present study generated data of more than 60× coverage with and without Cas9 telomeric labeling. The assembly results are shown in FIG. 7. The consensus maps from de novo assembly and the hg38 reference are shown as light blue bars and the dark blue vertical lines within these indicate the Nt.BspQI nick-label sites. The single molecules that comprise the respective consensus map are the yellow lines shown directly below the reference and consensus maps. The labels on the single molecules that align with the hg38 reference are dark green and those that do not align are light blue. The top consensus map were nick-labeled with Nt.BspQI and Cas9. The telomeres are the additional labels on the far left side of the single molecules (red T above) which are absent in the sample without Cas9. This clearly shows that Cas9 will specifically label the telomeric sites.

### Novel Subtelomeric Structures

As indicated in the elsewhere in Example 1 section, several new novel subtelomeric structures were discovered. The present study compared the novel structures with the other mapped genomes without telomere labeling. Similar structures are found through all mapped genomes. Some of them are as long as 1 Mb, and cannot be aligned to the hg38 reference. Based on the conserved pattern similar to 4p, it was believed that these novel structures belong to some of the unknown acrocentric chromosomes.

### Molecules for Telomere Length Measurement

The telomere length was measured for approximately 30 molecules for each chromosome arm of a sample. FIG. 8 shows the 32 molecules that were measured for 12p of LNCaP. The telomeres are designated as a red T. The consensus map aligned to the hg38 reference is shown above the nick-labeled single molecules after de novo assembly.

### Example 2: Single-Molecule Telomere Assay Via Optical Mapping (SMTA-OM) can Define the ALT Positivity of Cancer

Telomeres play an essential role in protecting the ends of linear chromosomes and maintaining the integrity of the human genome. One of the key hallmarks of cancers is their replicative immortality. As many as 85-90% of cancers activate the expression of telomerase (TEL+) as the telomere maintenance mechanism (TMM), and 10-15% of cancers utilize the homology-dependent repair (HDR)-based Alternative Lengthening of Telomere (ALT+) pathway. Here, the present study performed statistical analysis of the telomere profiling results from Single Molecule Telomere Assay via Optical Mapping (SMTA-OM), which is capable of quantifying individual telomeres from single molecules across all chromosomes. By comparing the telomeric features from SMTA-OM in TEL+ and ALT+ cancer cells, the present study demonstrated that ALT+ cancer cells display certain unique telomeric profiles, including increased fusions/internal telomere-like sequence (ITS+), fusions/internal telomere-like sequence loss (ITS−), telomere-free ends (TFE), super-long telomeres, and telomere length heterogeneity, compared to TEL+ cancer cells. Therefore, the present study propose that ALT+ cancer cells can be differentiated from TEL+ cancer cells using the SMTA-OM readouts as biomarkers. In addition, the present study observed variations in SMTA-OM readouts between different ALT+ cell lines that may be used as biomarkers for discerning subtypes of ALT+ cancer and monitoring the response to cancer therapy.

### Example 2-1

Human telomeres consist of unique repetitive DNA sequences, (TTAGGG)n, which are found at the terminal ends of each chromosome and function as protective barriers from the attrition of protein-coding regions. In normal somatic cells, telomere length is approximately 10-15 kilobases (kb), but over time, telomeres will inevitably shorten with each cell division in the absence of a telomere maintenance mechanism (TMM). In the case of neoplastic cells experiencing rampant proliferation, the vast majority of them initiate two TMMs, namely, the re-activation of telomerase (TEL+) and the adoption of the Alternative of Lengthening of Telomeres (ALT+) pathway, to avoid excessive shortening of telomeres, which could lead to cell cycle arrest, or senescence, or cell death. Approximately 10-15% of cancers lack detectable telomerase activity but are still capable of maintaining telomere integrity using the ALT pathway. Unlike human telomerase, which directly adds more telomere tract sequences to the pre-existing telomeres, the ALT+ cells rely on homology-dependent repair (HDR) to maintain their telomeres, which may result in unique structural changes at the telomeres and subtelomeres.

The ALT pathway is more prevalent in certain types of cancer, including neuroblastoma (NB), pancreatic neuroendocrine tumors (PanNET), osteosarcoma, and glioma. For some, such as the PanNET, the ALT positivity predicts a likely worse prognosis. However, for others, such as NBs, the opposite is often true. Mechanistically, DNA damage response (DDR) and HDR proteins play an essential role in the ALT pathway. Recent studies from multiple groups have shown that a unique HDR pathway, the Break-Induced Replication (BIR) pathway, is critical for the ALT. The potential templates for the BIR include the telomere from the homologous chromosome, from a nonhomologous chromosome, or even from the extrachromosomal telomeric repeats (ECTRs), which are abundant in the ALT+ cells. Identification of the TEL+ cells primarily relies on the well-established telomerase repeated amplification protocol (TRAP) assay. However, the identification of the ALT+ cells is much more tedious and has to rely on the positivity of multiple assays, including telomere fluorescent in situ hybridization (Telo-FISH), ALT-associated acute promyelocytic leukemia bodies (APBs), telomere dysfunction induced foci (TIFs), telomere sister chromatin exchange (tSCE), C-circle, and others. Most recently, using machine learning and whole genome sequencing (WGS), Lee and colleagues proposed that telomere variants may potentially be used to differentiate ALT+ cells from TEL+ cells because they are generated from distinct mechanisms. Therefore, novel and more reliable biomarkers/assays are urgently needed for ALT research as well as for identifying the ALT+ tumors in the clinic for targeted therapy in the near future.

Irrespective of ALT+ or TEL+ cells, detecting and quantifying the genome-wide changes of telomeres at individual chromosome arms have been challenging. Current assays that have been used for this purpose include Q-PCR (quantitative polymerase chain reactions), Q-FISH (quantitative fluorescent in situ hybridizations), STELA (single telomere length analysis), and TeSLA (telomere shortest length assay). As discussed in detail by Lai and colleagues, every one of these approaches has its limitations and is not easily applicable to genome-wide characterization of telomeres for both TEL+ and ALT+ cells.

A novel method that enables genome-wide analysis of telomeres at the single-molecule level, called Single-Molecule Telomere Analysis via Optical Mapping (SMTA-OM) technology was developed (McCaffrey et al., Genome Res. 2017; 27:1904-1915). As whole-genome optical mapping can identify each subtelomeric region adjacent to the telomeres, SMTA-OM assay can identify and characterize each telomere and its associated features. Using the SMTA-OM, the present study analyzed the telomere/chromosome end status of two TEL+ cancer cell lines (LNCaP and UMUC3) as well as a senescent primary lung fibroblast cell line, IMR90-S. Subsequently, the present study utilized the SMTA-OM to characterize the telomere/chromosome end status of three ALT+ cell lines (Saos-2, SK-MEL-2, and U2OS). From these two recent studies, the present study demonstrated that the SMTA-OM can be used to visualize and quantify telomeres at the single-molecule level of a specific chromosome arm and with a wide range in length, from 100 base pairs (bp) to over 100 kilobases (kb). Other telomere/chromosome end features that can be detected and quantified by the SMTA-OM include ECTRs and telomere-free ends (TFEs). Most intriguingly, the SMTA-OM is particularly effective in detecting and quantifying the telomere/chromosome end fusion events in the ALT+ cells. The detected fusions include the fused molecules with internal telomere-like sequences (fusion/ITS+) and fused molecules with no detectable internal telomere-like sequences (fusion/ITS−).

Here, the present study further analyzed the SMTA-OM results in both ALT+ and TEL+ cancer cells as well as the IMR90-S cells and demonstrated that the SMTA-OM alone can potentially be used to define the ALT positivity through the following readouts/parameters: (1) the presence and abundance of fusion/ITS+; (2) the presence and abundance of the fusion/ITS−; (3) the presence and abundance of TFEs; (4) the presence and abundance of super-long telomeres; and (5) the heterogeneity of telomere lengths at the whole-genome level. The present study proposes that these SMTA-OM readouts can be used to identify the ALT positivity in tumors in the clinic for targeted therapy.

### Example 2-2: Materials and Methods

### Cell Preparation and High Molecular Weight DNA Extraction

The three ALT+ cell lines, U2OS, Saos-2, and SK-MEL-2, were purchased from American Type Culture Collection (ATCC). Both U2OS and Saos-2 were cultured in McCoy's 5a medium supplemented with 10% and 15% fetal bovine serum (FBS), respectively. SK-MEL-2 was cultured in Eagle's Minimum Essential Medium supplemented with 10% FBS. UMUC3 was cultured in Eagle's Minimum Essential Medium containing Earle's salts, nonessential amino acids (NEAA), and L-glutamine (2 mM) with 10% FBS. LNCaP cells were purchased from ATCC and cultured in RPMI 1640 with 2 mM L-glutamine, 10 mM HEPES, 1 mM sodium pyruvate, 4500 mg/L glucose, and 1500 mg/L sodium bicarbonate and 10% FBS. IMR90-S cells were purchased from Coriell Cell Repository and cultured in EMEM with Earle's salts, NEAA, and 2 mM L-glutamine supplemented with 15% FBS. Cells were passaged using 0.25% trypsin-EDTA, except for IMR90-S, which was passaged with 0.05% Trypsin-EDTA. High molecular weight DNA extraction, guide RNA (gRNA) preparation, and the two-color DNA labeling process were conducted as described in McCaffrey et al. (Genome Res. 2017; 27:1904-1915).

### Loading and Imaging of DNA

The DNA samples were treated with Protease and IrysPrep Stop Solution (BioNano Genomics, San Diego, CA, USA). The labeled DNA was stained with YOYO-1 (Invitrogen, Carlsbad, CA, USA) before being loaded into the nanochannels following an established procedure. A total of 180 Gb data, which is about 60× coverage, provided around 30 molecules with telomeres for each chromosome arm. The image analysis was performed following the established method (McCaffrey et al. Genome Res. 2017; 27:1904-1915).

### Telomere Length Analysis

Telomeres were identified as the extra labels at the end of a DNA molecule or in the middle of a fused DNA molecule. The fluorescence intensity was used to infer the length of each individual telomere as described in the established protocol (McCaffrey et al. Genome Res. 2017; 27:1904-1915).

### Example 2-3: Results

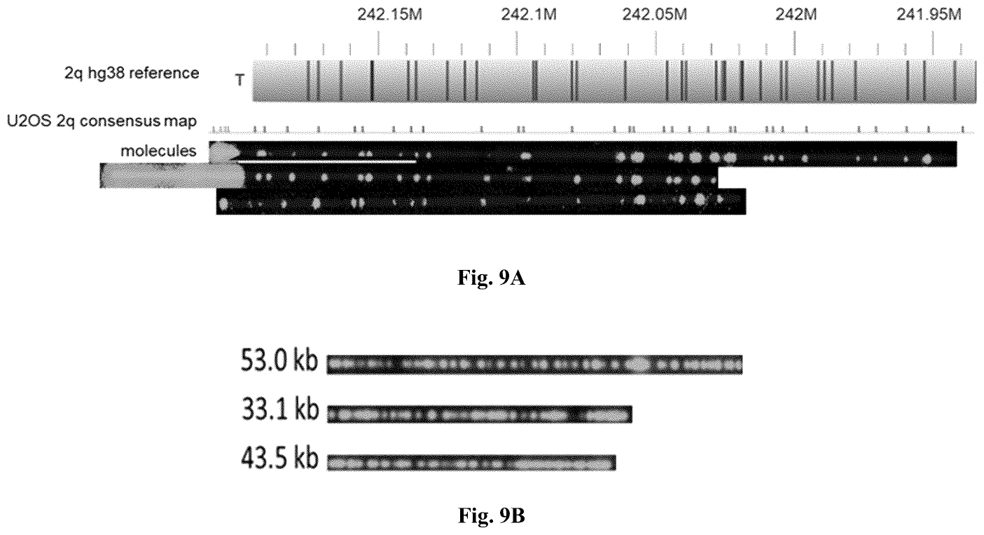

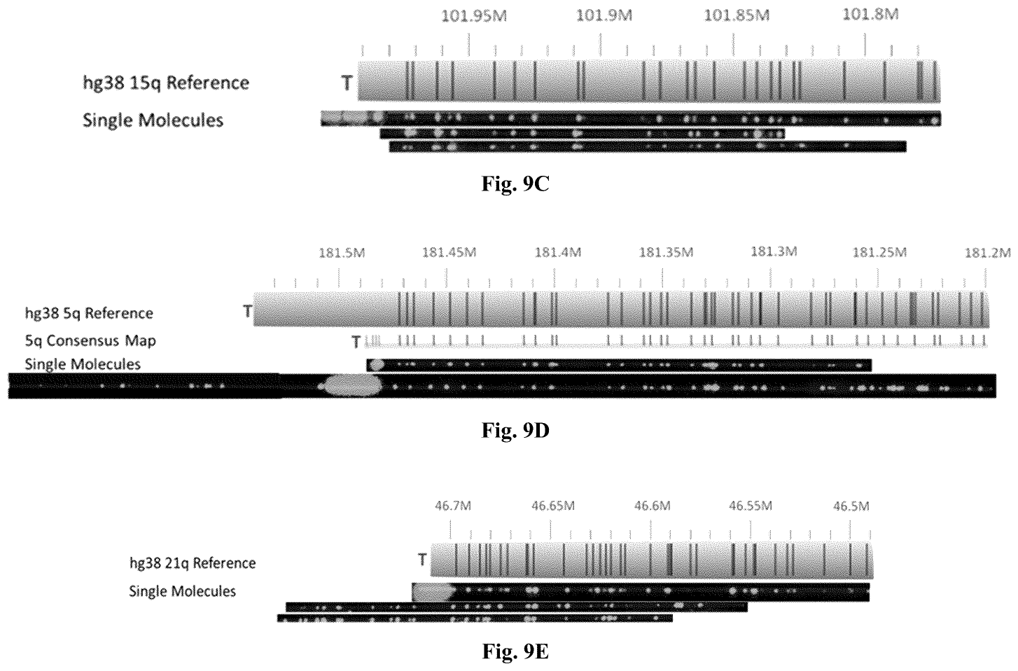

In SMTA-OM, first developed by McCaffrey et al., high-molecular-weight genomic DNA (gDNA) molecules were first nicked by a nickase, Nt. BspQI, at recognition sequence 5′-GCTCTTC-3′. The nick sites across the whole genome were then tagged with a green fluorophore by Taq DNA polymerase. An in vitro CRISPR/Cas9 sgRNA-directed nickase system then directed the specific labeling of telomeric DNA, with the same-colored fluorophore also by Taq DNA polymerase. All DNA molecules were also stained with YOYO-1 (blue). The two-colors-labeled long chromosomal fibers (>150 kilobases (kb)) were linearized in the NanoChannel Arrays (purchased from Bionano Genomics) and imaged. FIGS. 9A-9E show the representative images of the two-colors-labeled telomeres/chromosome ends and ECTRs from U2OS cells.

### Example 2-3-1: Summary of Telomere/Chromosome End Characterization Using SMTA-OM

Out of 46 chromosome arms, the present study was able to measure molecules from 33 arms of SK-MEL-2, 28 arms of Saos-2, 34 arms of U2OS, 34 arms of LNCaP, 28 arms of UMUC3, and 27 arms of IMR90-S. Chromosome arms that could not be measured were due to a lack of either reference or assembled consensus contigs covering the regions of interest. Raw measurements were collected as outlined by McCaffrey et al. (Genome Res. 2017; 27:1904-1915).

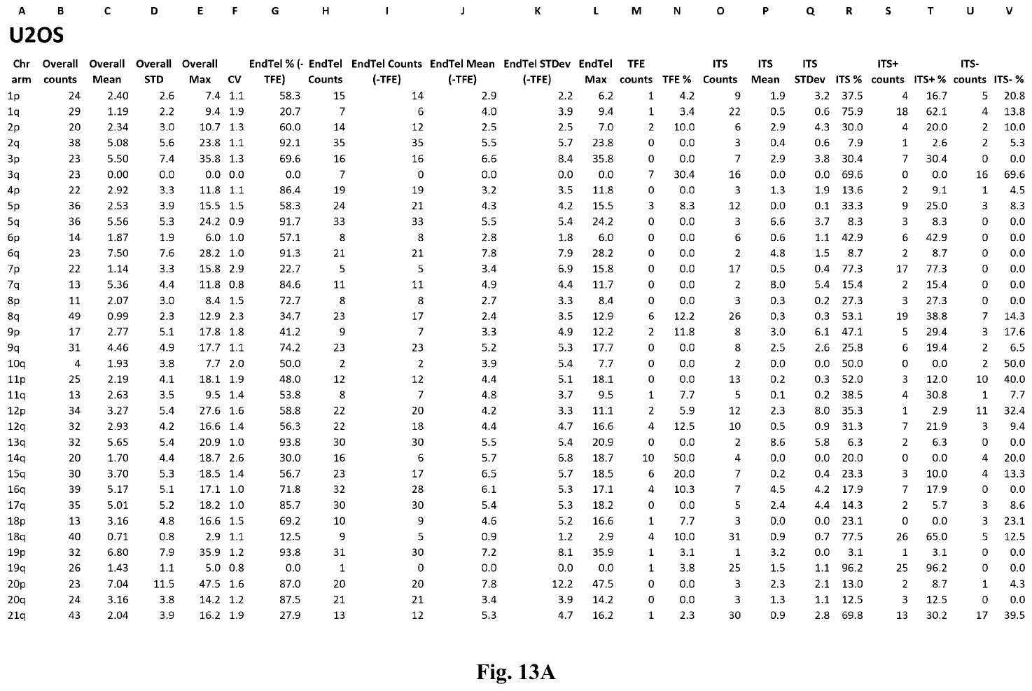

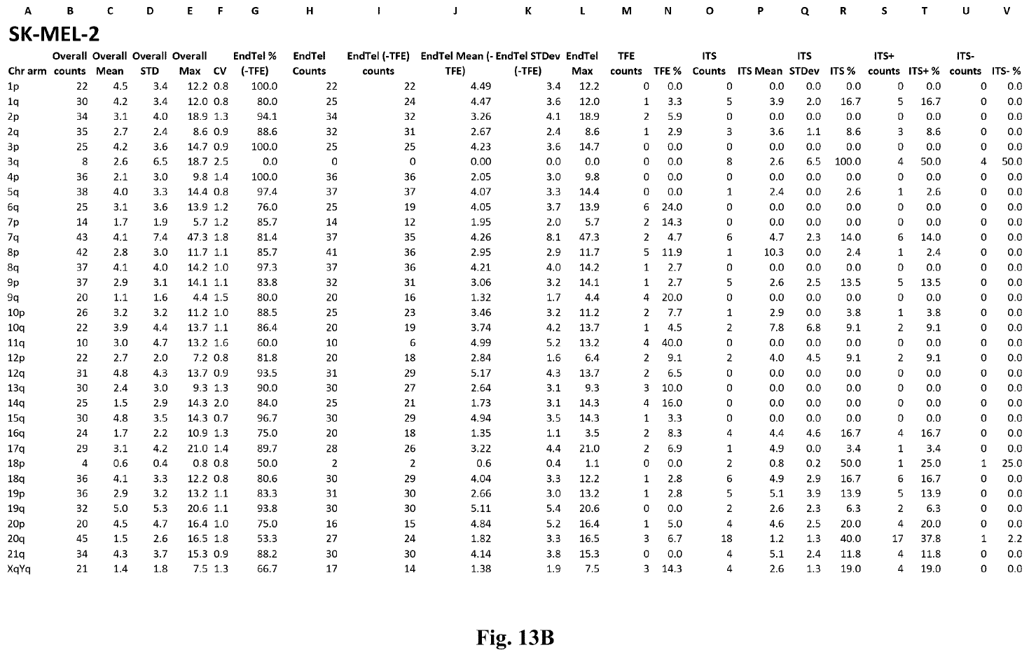

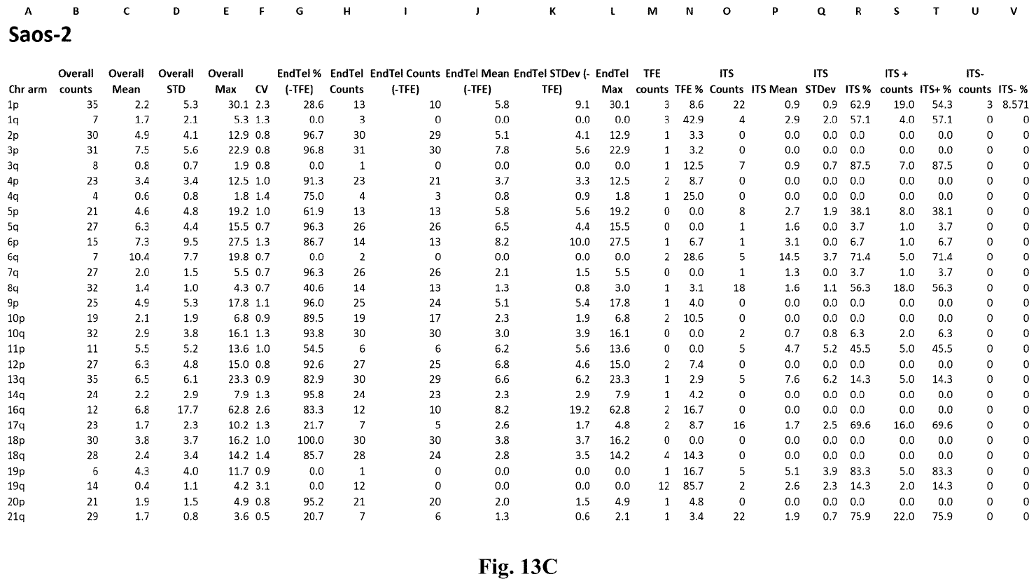

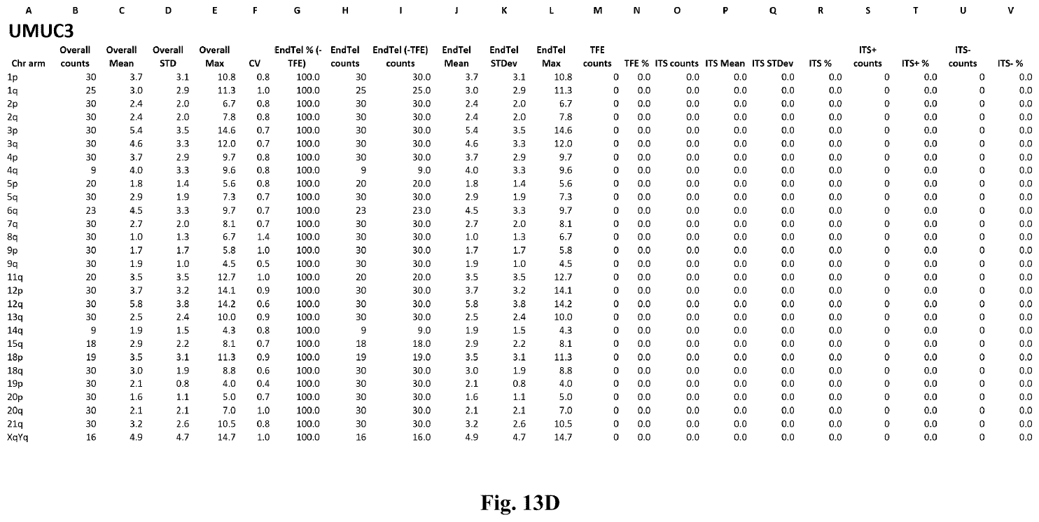

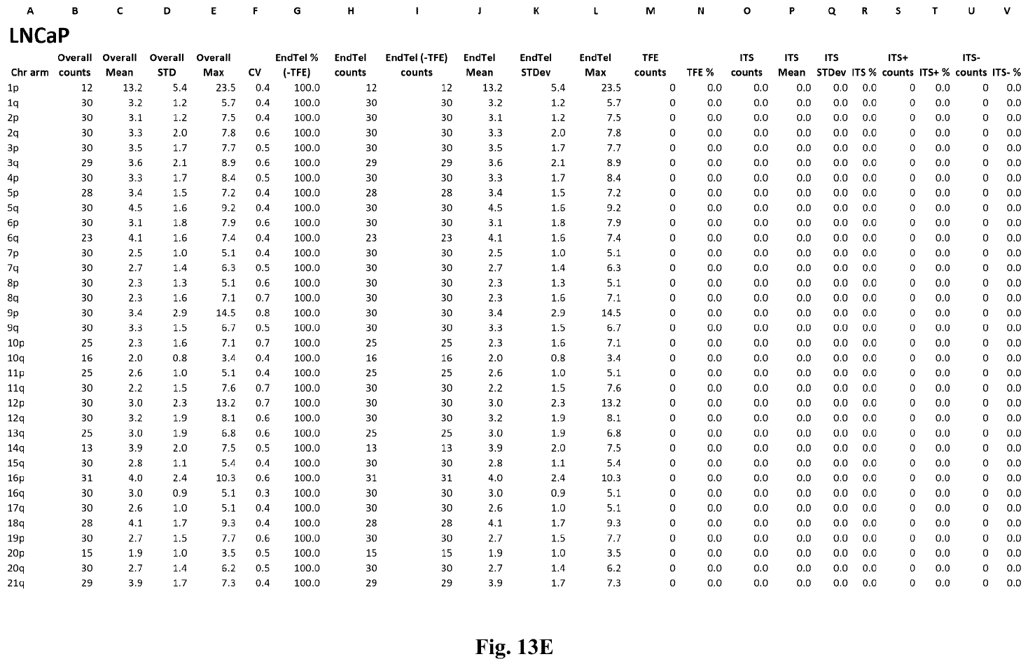

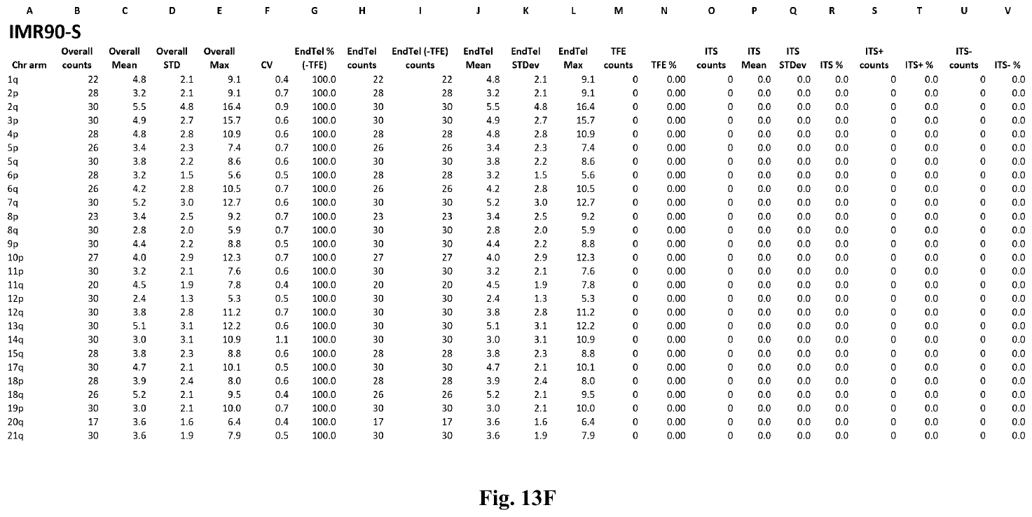

Mean telomere lengths, standard deviations, and percentages of molecules with TFE, ITS+, and ITS− for each analyzed chromosome arm were calculated for each cell line (FIGS. 13A-13F). The overall count column includes the total number of molecules analyzed for a specific arm. EndTel counts (−TFE) includes the total number of molecules with end telomeres while excluding those with TFEs. TFE count is the total number of molecules without any detectable telomere signal, but the labeling pattern aligns with the reference in the subtelomere region. ITS (the sum of ITS+ and ITS−), ITS+, and ITS− counts are determined in the same manner as mentioned above for the other parameters.

Next, to evaluate the mean telomere length of each arm from the end telomeres, the present study summed measurements from a chromosome arm and divided them by the corresponding count (FIGS. 13A-13F). The same method was used to calculate the average lengths of ITS+ molecules. The longest measured telomere for each arm is also listed (column E, labeled as Overall Max).

The present study was also interested in the frequency of TFE, ITS+, and ITS− occurrence in each chromosome arm per cell line (FIGS. 13A-13F, columns N, T, V). The percentage for EndTel (−TFE) was calculated by dividing the number of molecules with end telomeres by the total number of molecules for an individual chromosome arm and multiplying by 100. TFE %, ITS %, ITS+%, and ITS−% were all calculated in a similar manner.

All the mentioned calculations were completed for the IMR90-S cells, the three ALT+ cell lines and the two TEL+ cell lines.

### Example 2-3-2: ALT+ Cells Manifest Elevated Fusion/ITS+ and Fusion/ITS−

ALT+ cells can be recognized by their unique characteristics of the telomeric/chromosome end regions, such as the elevation of fusion/ITS+ and fusion/ITS−, which are the unique characteristics of ALT+ cells (U20S, SK-MEL-2, and Saos-2) compared to TEL+ cells (UMUC3, LNCaP) and IMR90-S. Approximately 9% of SK-MEL-2, 19% of Saos-2, and 35% of U20S molecules are fusions (Table 1). Strikingly, LNCaP, UMUC3, and senescent IMR90 are completely absent of the fusions. On average, ITS+ was observed in 8.6% of all SK-MEL-2, 18% of all Saos-2, and 23% of all U2S molecules. Although the amount of fusion/ITS− in ALT+ cancer cells is relatively lower, it is still elevated in all three ALT+ cancer cells. Fusion/ITS− was recorded in roughly 0.500 of all SK-MEL-2, 0.5% of all Saos-2, and 12% of all U2OS molecules (Table 1).

TABLE 1
Summary of SMTA-OM readouts for ALT+ (U2OS, Saos-2, SK-MEL-2), TEL+
(UMUC3, LNCaP), and IMR90-S cells. It includes overall telomere mean length, end telomere
mean length excluding the telomere-free ends (−TFE), fusion/internal telomere-like signal
(ITS+), mean telomere length, heterogeneity of telomere length represented by the coefficient of
variation (CV), the percentage of fusion/ITS+ molecules, the percentage of fusion/internal
telomere-like signal loss (ITS−) molecules, the percentage of telomere-free ends (TFEs), and the
status of the extrachromosomal telomeric repeats (ECTR). N/A indicates no fusion/ITS+
observed in a cell line.

| | ALT+ | | | TEL+ | | Control |
|---|---|---|---|---|---|---|
| | U2OS | Saos-2 | SK-MEL-2 | UMUC3 | LNCaP | IMR90-S |
| Overall telomere | 3.4 ± 5.1 | 3.8 ± 5.2 | 3.2 ± 3.8 | 3.1 ± 2.8 | 3.2 ± 2.2 | 4.0 ± 2.6 |
| mean length (kb) | | | | | | |
| End telomere (−TFEs) | 5.0 ± 2.2 | 4.5 ± 5.6 | 3.5 ± 3.9 | 3.1 ± 2.8 | 3.2 ± 2.1 | 4.0 ± 2.6 |
| mean length (kb) | | | | | | |
| Fusion/ITS+ mean | 1.8 ± 3.3 | 2.7 ± 3.6 | 3.7 ± 3.4 | N/A | N/A | N/A |
| telomere length (kb) | | | | | | |
| The heterogeneity of | 1.4 | 1.2 | 1.1 | 0.8 | 0.5 | 0.6 |
| telomere length (CV) | | | | | | |
| Fusion/ITS+ (%) | 23.1 | 17.6 | 8.6 | 0 | 0 | 0 |
| Fusion/ITS− (%) | 12.2 | 0.5 | 0.5 | 0 | 0 | 0 |
| Telomere Free Ends | 6.4 | 6.3 | 7.6 | 0 | 0 | 0 |
| (TFEs) (%) | | | | | | |
| ECTR | High | High | High | No data | No data | No data |

The present study then calculated the fusion/ITS+ and fusion/ITS− percentage of each individual arm of ALT+ cells (FIGS. 13A-13F, columns T, V). Fusion/ITS+ and fusion/ITS− percentages vary greatly among individual arms of the three ALT+ cells. For example, 50% of 3q molecules from SK-MEL-2, 87.5% of 3q molecules from Saos-2, and 96.2% of 19q from U2OS are fusion/ITS+, while some chromosome arms, such as 3q from U2OS and 2p from both SK-MEL-2 and Saos-2, have no fusion/ITS+ molecules. For fusion/ITS−, the chromosome arms with the greatest elevation of fusion/ITS− from each ALT+ cell were 1p from Saos-2 (8.6%), 3q from SK-MEL-2 (50%), and 3q from U2OS (69.6%). Most of the chromosome arms from the three ALT+ cells are without fusion/ITS−. Overall, chromosome arm 3q is the most unique chromosome arm with respect to the formation of fusion. For SK-MEL-2 cells, all the molecules containing chromosome arm 3q are fusions (50%—fusions/ITS+; 50%—fusion/ITS−). For Saos-2 cells, the chromosome arm 3q has the most fusions and all of them are fusion/ITS+(87.5%). For U2OS cells, all the fusions containing chromosome arm 3q are fusion/ITS− (69.6%), which rank it the highest in fusion/ITS− and the sixth highest in total number of fusions. At present, the reason why the chromosome arm 3q is so prone to form fusions is unknown.

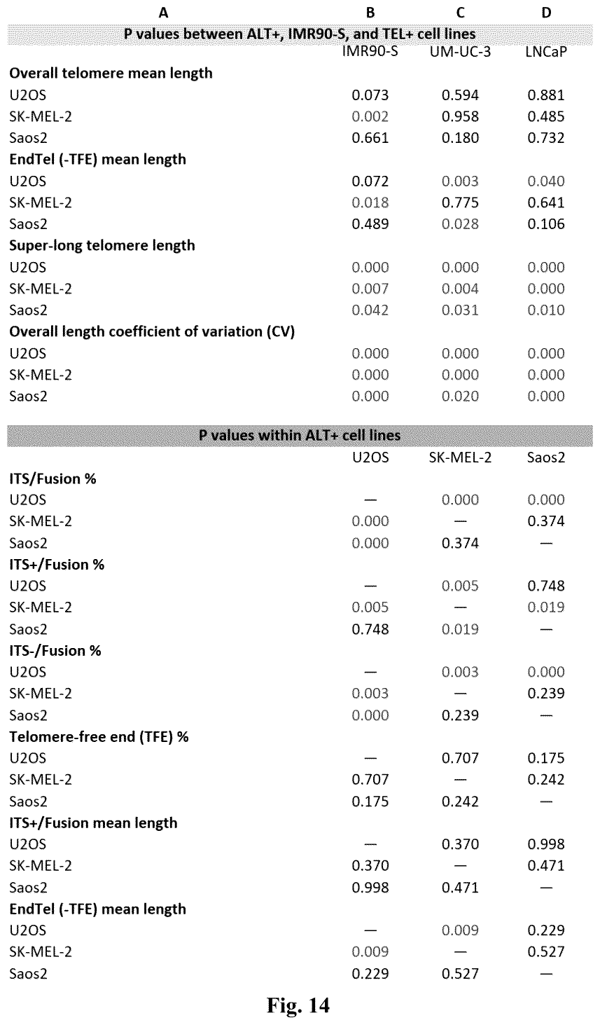

Within the three ALT+ cell lines, the percentages of fusion/ITS+ of each chromosome arm (FIGS. 13A-13F, column T) were used to perform a two-tailed t-test to evaluate whether the fusion/ITS+ percentage is significantly different between the ALT+ cell lines (FIG. 14). For fusion/ITS+, the present study found U2OS and SK-MEL-2, as well as Saos-2 and SK-MEL-2, to be significantly different from each other, with p values of 0.005 and 0.019, respectively (FIG. 14, C27, C29). There was no statistical difference between fusion/ITS+ percentages of Saos-2 and U2OS (p=0.748). The same procedure was followed for fusions/ITS− (FIGS. 13A-13F, column V). U20S has a significantly different frequency of fusion/ITS− compared to both SK-MEL-2 and Saos-2 (p=0.003, 0.000, respectively) (FIG. 14, B32 and B33). In contrast to fusion/ITS+, Saos-2 and U20S have significantly different percentages of fusion/ITS− (p=0.000); however, there was no significant difference in fusion/ITS− when comparing Saos-2 and SK-MEL-2 (p-value=0.239) (FIG. 14, B33, C33, respectively).

Taken together, the data suggest that the fusions (both fusion/ITS+ and fusion/ITS−) not only can be used to distinguish ALT+ cells from TEL+ cells; they could also be useful for differentiating the subgroups of ALT+ cancers.

### Example 2-3-3: ALT+ Cells Show Elevated Telomere-Free Ends

The elevation of TFEs in ALT+ cells is another pronounced feature compared to TEL+ cells and senescent IMR90 (Table 1 and FIGS. 13A-13F, Columns M and N). SK-MEL-2 has the greatest frequency of TFEs at 7.6%. This was followed by U2OS and Saos-2 at 6.4% and 6.3% of molecules with TFEs, respectively. In stark contrast, neither LNCaP, nor UMUC3, nor senescent IMR90 cells had any detectable TFEs. Intriguingly, the present study observed that certain chromosome arms in ALT+ cells are more likely to experience complete telomere loss than others. The chromosome arms in the SK-MEL-2 with the highest elevation of TFEs were 11q (40%), 6q (24%), and 9q (20%). The arms 19q (85.7%), 1q (42.9%), and 6q (28.6%) had the greatest TFEs from Saos-2. The top three arms with TFEs for U2OS were 14q (50.0%), 3q (30.4%), and 12q (12.5%). Both SK-MEL-2 and Saos-2 have a high incidence of TFEs from chromosome arm 6q. Only one chromosome arm, 5q, was absent of any TFEs from U2OS, SK-MEL-2, and Saos-2. The elevation of TFEs is a characteristic unique to ALT+ cancer cells, since no TFE was recorded for the two TEL+ cells (UMUC3 and LNCaP) and the senescent IMR90. These data indicate that there is heightened spontaneous DNA damage in the subtelomeric regions in all the ALT+ cells, resulting in the complete loss of telomeres.

Within the three ALT+ cells, the percentages of TFEs from each chromosome arm were used to perform a two-tailed t-test to evaluate whether the TFE percentage is significantly different between the ALT+ cell lines (FIG. 14). The t-test reveals no statistical difference in TFE percentage between U2OS, SK-MEL-2, and Saos-2 cell lines.

### Example 2-3-4: Overall Telomere Mean Length and End Telomere (−TFEs) Mean Length Manifest Either Minimal or No Differences Between the ALT+ Cells and TEL+ Cells

### Overall Telomere Mean Length

The overall telomere mean lengths include measurements from end telomeres, TFEs, ITS+, and ITS−. As shown in Table 1, the overall telomere mean lengths for ALT+ samples were 3.8±5.2 kb for Saos-2, 3.4±5.1 kb for U2OS, and 3.2±3.8 kb for SK-MEL-2. TEL+ cells had overall telomere mean lengths of 3.2±2.2 kb for LNCaP and 3.1±2.8 kb for UMUC3. The overall telomere mean length for IMR90-S is 4.0±2.6 kb. Using the overall telomere mean lengths from all six cell lines, a two-tailed unequal variance t-test was performed to evaluate the statistical significance between ALT+ and TEL+ cell lines. Intriguingly, considering most overall telomere mean lengths are within 1 kb of each other across the cell lines, there is little to no difference seen among the ALT+, TEL+, and IMR90-S cells, apart from SK-MEL-2 and IMR90-S at p=0.002 (FIG. 14, B5). Therefore, the present study concludes that the overall telomere mean length is not a good indicator for differentiating ALT+ cells from TEL+ cells. Overall telomere mean lengths include a mixture of molecules with TFEs and ITS−, which were assigned a length value of 0 kb, and thus will skew the average telomere length.

The present study observed a wide distribution of telomere lengths for ALT+ cells and high variability across individual chromosome arms (FIGS. 13A-13F), suggesting that, indeed, the lengths of telomeres in ALT+ cells are more heterogeneous than those in the TEL+ cells. Chromosome arm 20p is the only arm where U2OS (7.0±11.5 kb), SK-MEL-2 (4.5±4.7 kb), and Saos-2 (1.9±1.5 kb) all contain telomeres with a longer average length than that of UMUC3 (1.6±1.1 kb) and slightly longer than LNCaP (1.9±1.0). The 20p arm from senescent IMR90 cells was not analyzed, so it is not certain whether the overall telomere mean length would also be less than that of the ALT+ cells. There are no chromosome arms where the overall telomere mean lengths of UMUC3, LNCaP, and senescent IMR90 cells were all longer than U2OS, SK-MEL-2, and Saos-2 cells overall telomere mean lengths.

### End Telomere (−TFEs) Mean Length

As mentioned above, the overall telomere mean length of ALT+ cancer cells is skewed toward shorter lengths due to the presence of many TFE and ITS− molecules. The end telomere (−TFEs) mean length, which excludes the TFE and ITS− molecules, may be a better differentiating parameter. As shown in Table 1, the end telomere mean lengths (excluding TFEs and ITS−) for the three ALT+ cells were 5.0±2.2 kb for U2OS, 4.5±5.6 kb for Saos-2, and 3.5±3.9 kb for SK-MEL-2. TEL+ cells and IMR90-S retained the same measurements of 3.2±2.2 kb for LNCaP, 3.1±2.8 kb for UMUC3, and 4.0±2.6 kb for senescent IMR90, since no TFEs or ITS is recorded in these cells. Using all individual end telomere (−TFEs) mean lengths, a two-tailed t-test was conducted to evaluate the significance of the end telomere mean length excluding TFEs and ITS− between ALT+ and TEL+ cell lines (FIG. 14). The present study found that U20S is statistically different from both UMUC3 and LNCaP, with p values of 0.003 and 0.040, respectively (FIG. 14, C8, D8). However, U20S is not significantly different from senescent IMR90 (p=0.072) (FIG. 14, B8). The only ALT+ cell line found to be significantly different from senescent IMR90 is SK-MEL-2 (p=0.018) (FIG. 14, B9). No other combinations of ALT+ and TEL+ cells resulted in significant p values (FIG. 14). Among the three ALT+ cells studied, only U20S and SK-MEL-2 significantly varied in end telomere (−TFEs) mean length (p=0.009) (FIG. 14, B44). Therefore, the data indicate that end telomere mean length excluding TFEs and ITS− is not a distinctive feature of ALT+ cells either.

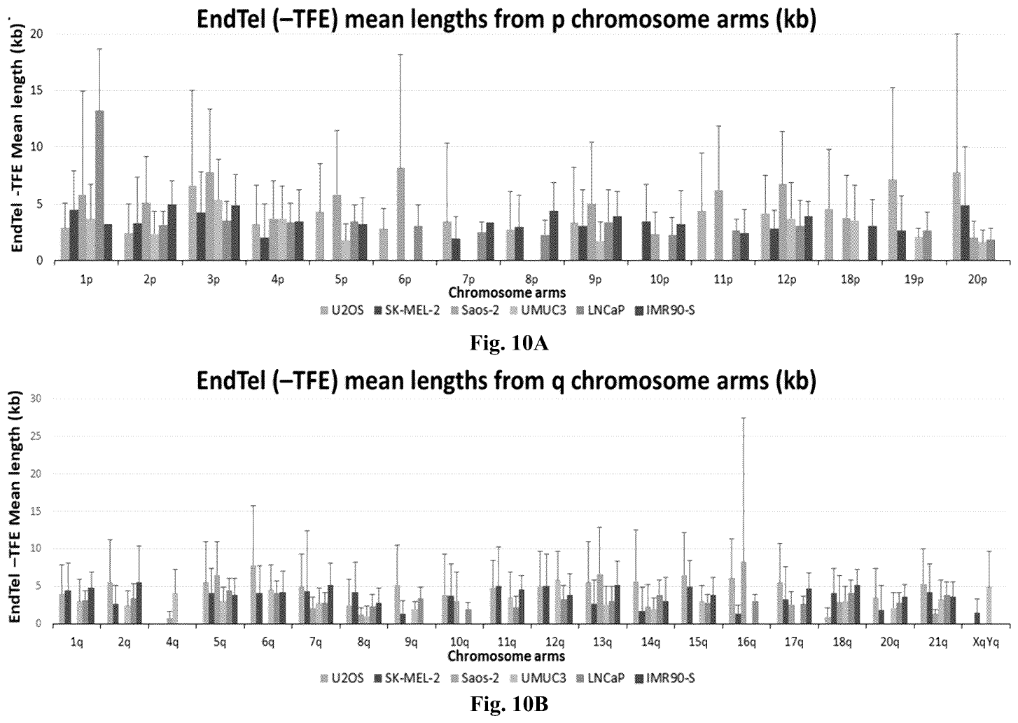

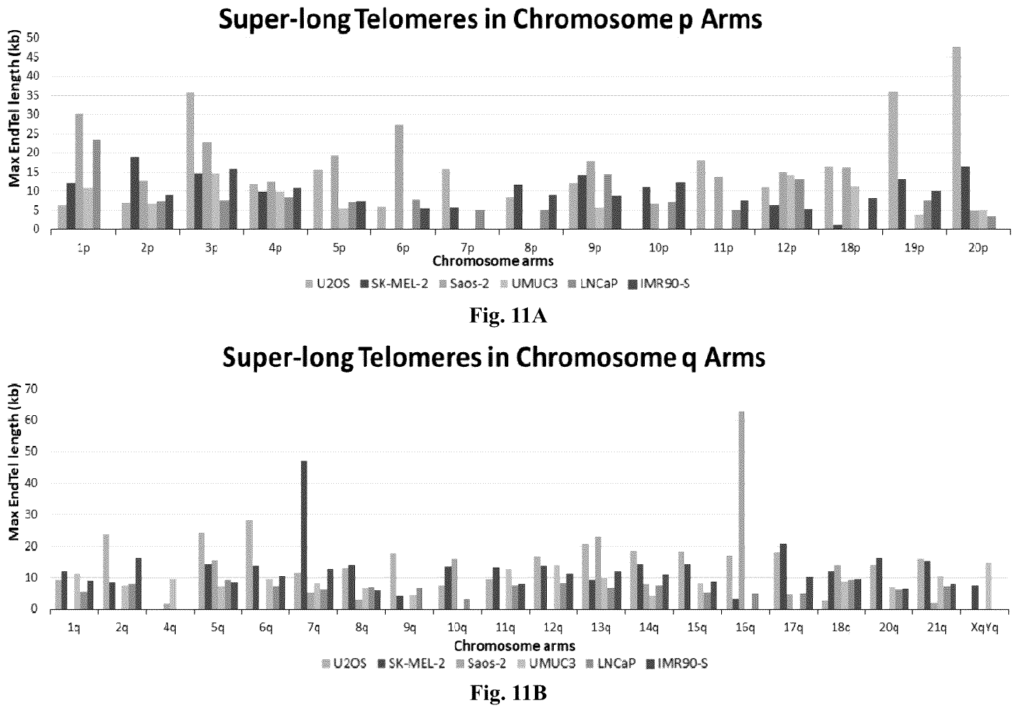

End telomere (−TFE) mean lengths are also visualized in two separate bar graphs representing p and q chromosome arms in FIGS. 10A-10B. The chromosome arms depicted have at least 1 ALT+ cell line and 1 TEL+ cell line for comparison. Certain chromosome arms in ALT+ cells had substantially longer telomeres compared to the corresponding chromosome arm from the TEL+ samples. Similar to the overall telomere mean lengths, 20p is the only arm where all three ALT+ cell lines have end telomere (−TFE) mean lengths greater than the two TEL+ cells and the senescent IMR90, but there are no arms where the TEL+ cells have greater end telomere (−TFE) mean lengths than all three ALT+ cell lines. Taken together, the data indicate that the end telomere (−TFE) mean length for the individual arm is not a useful readout to differentiate ALT+ cells from TEL+ cells either.

### Example 2-3-5: ALT+ Cells Have More Super-Long Telomeres

Interestingly, the present study observed that the ALT+ cells have more super-long telomeres. As seen in FIGS. 11A-11B, the present study compiled the longest telomere of each chromosome arm of all six cells and plotted them in a bar graph. Super-long telomeres from p and q chromosome arms in all six cell lines are represented by separate bar graphs. A molecule of 16q from Saos-2 has the longest telomere length of 62.8 kb, while in U20S and SK-MEL-2, the longest telomeres are molecules from 20p of U20S with 47.5 kb and 7q from SK-MEL-2 at 47.3 kb, respectively. The longest telomeres of each chromosome arm in ALT+ cells are generally from end telomeres, not from fusion/ITS+. For comparison, the longest telomere from UMUC3 is 14.75 kb from XqYq, the longest telomere from LNCaP is 23.5 kb from 1p, and the longest telomere from senescent IMR90 is 16.4 from 2q.

Out of 38 total chromosome arms analyzed, U20S has the most chromosome arms (14) with the longest telomeres. These 14 arms are 2q, 3p, 5q, 6q, 7p, 9q, 11p, 12q, 14q, 15q, 18p, 19p, 20p, and 21q. SK-MEL-2 had 10 chromosome arms with the longest telomeres, including 1q, 2p, 7q, 8p, 8q, 10p, 11q, 17q, 19q, and 20q. The same as SK-MEL-2, Saos-2 has the longest telomere for 10 chromosome arms also, which includes 1p, 4p, 5p, 6p, 9p, 10q, 12p, 13q, 16q, and 18q. Note that chromosome arms 3q, 4q, 16p, and XqYq either lack a sufficient number of measurements for analysis or have very few end telomeres in all the ALT+ cells.

A two-tailed t-test using the longest telomeres of each chromosome arm among five cell lines clearly shows that ALT+ cells have statistically more super-long telomeres than TEL+ cells (FIG. 14). Overall, the present study observed many more super-long telomeres in ALT+ cells. Telomeres longer than 15 kb were recorded for 4.3% of Saos-2, 4.1% of U20S, and 1.0% of SK-MEL-2. In contrast, 0.4% of LNCaP telomeres and 0.3% of IMR90 senescence telomeres are longer than 15 kb, while UMUC3 had no telomeres measuring longer than 15 kb. Therefore, the data indicate that the presence of super-long telomeres may also serve as an indicator for differentiating ALT+ from TEL+ cells.

### Example 2-3-6: ALT+ Cells Show Higher Telomere Length Heterogeneity

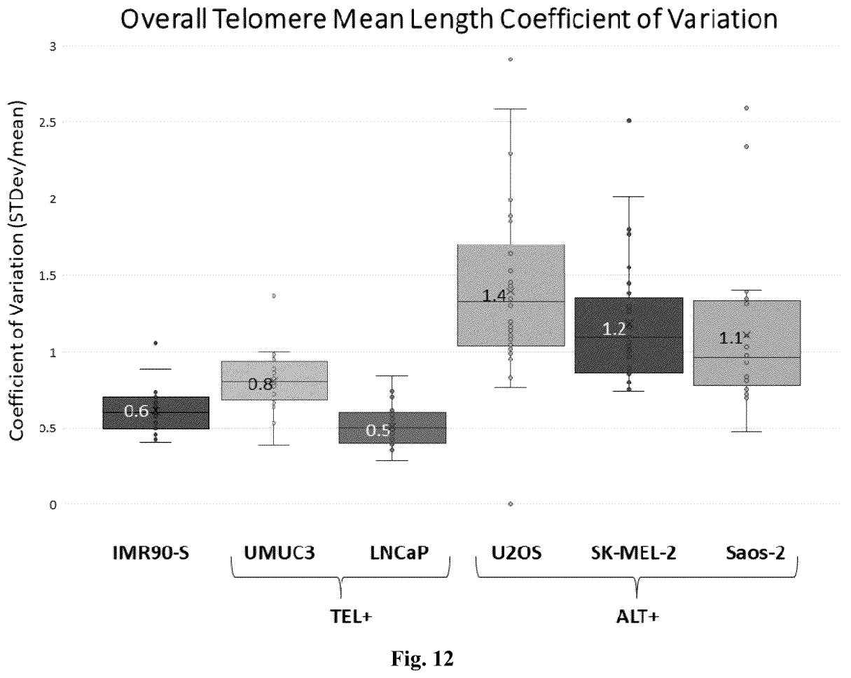

As mentioned above, the higher telomere length heterogeneity in ALT+ cells is evident. Next, the present study calculated the overall telomere mean length of all telomeres across all chromosome arms (including end telomeres, TFEs, ITS+, and ITS−) and associated standard deviations (Table 1). For example, U20S and UMUC3 have an overall mean length and standard deviations (STD) of 3.4 kb±5.1 kb and 3.1 kb±2.8 kb, respectively. ALT+ cells typically have higher STDs. To further quantify the heterogeneity, the present study calculated the coefficient of variation (CV) of the overall telomere mean length. CV is calculated by dividing the standard deviation by the mean. As shown in FIG. 12, the CV values are all above 1 for the three ALT+ cells, while the CVs are all less than 1 for the two TEL+ cells and senescent IMR90, which reflects a much wider distribution of telomere lengths in the three ALT+ cells. Several factors contribute to the elevated CVs in ALT+ cells. First, ALT+ cells have more super-long telomeres. Second, ALT+ cells have increased ITS− and TFEs, whose telomeres are recorded as 0 kb. Finally, telomere measurements in ITS+ are generally shorter compared to end telomeres.

The present study also investigated the CV values for individual chromosome arms. The present study calculated the overall telomere mean length of each chromosome arm and its associated standard deviation (FIGS. 13A-13F, Column F). For example, the overall telomere mean length and standard deviation of 1p for U2OS and UMUC3 are 2.4±2.6 kb and 3.7±3.1 kb, respectively. In U2OS cells, the maximum overall telomere mean length standard deviation is 11.5 kb from 20p while the minimum is 0.0 kb from 3q since all the molecules are fusion/ITS−. The CVs of each chromosome arm for all six cell lines are summarized in FIGS. 13A-13F. Senescent IMR90 and the two TEL+ cells (UMUC3 and LNCaP) show a narrow distribution with a median CV of less than 1, with two exceptions (8q of UMUC3, CV=1.36; 14q of IMR90-S, CV=1.05). In stark contrast, the large majority of chromosome arms of the three ALT+ cells have a much wider CV distribution with median CVs greater than 1.

To compare the heterogeneity of telomeres of the two samples, the present study performed a two-tailed t-test, concluding that the difference in CV between all combinations of ALT+ and TEL+ cells is statistically significant (FIG. 14). Taken together, the data indicate that telomere length heterogeneity, at both the genome level as well as the individual chromosome arm level, is a very pronounced feature of the ALT+ cells.

### Example 2-4

Here, the present study performed an in-depth comparative analysis of the SMTA-OM results from three ALT+ cells (U2OS, Saos-2, and SK-MEL-2), two TEL+ cells (UMUC3 and LNCaP), and the non-transformed but senescent IMR90 (IMR90-S) cells. The results are summarized in Table 1. “N/A” in Table 1 indicates that there are no data relevant to the fusion/ITS+ mean telomere length since there is no fusion/ITS+ molecule detected in the two TEL+ cells and IMR90-S cells. ECTRs were detected and analyzed in the three ALT+ cells but were not detected in the two TEL+ cells and the IMR90-S cells. In addition, there were very few molecules from the short chromosome arms (i.e., the p arms) of all five acrocentric chromosomes (chromosomes 13, 14, 15, 21, 22) that were detected by the SMTA-OM. Therefore, chromosome arms 13p, 14p, 15p, 21p, and 22p were excluded from the analysis for all six cell lines.

The data herein indicate that there is no significant difference among the six cell lines for the overall telomere mean lengths except between SK-MEL-2 and IMR90-S(p=0.002) (FIG. 14). The higher prevalence of TFEs and fusion/ITS− found in the ALT+ cells may have obscured the difference. TFEs and fusion/ITS− are recorded as a length of 0 kb in the analysis, which will decrease the overall telomere mean length. In addition, the fusion/ITS+, while it has a measurable telomere signal, tends to be shorter compared to the end telomeres. Therefore, the present study excluded TFEs and fusions/ITS− when calculating the end telomere (−TFE) mean length. The end telomere (−TFE) mean length of U2OS is significantly longer than that of UMUC3 and LNCaP (p=0.003, 0.040, respectively) (FIG. 14). The end telomere (−TFE) mean length in SK-MEL-2 is significantly longer compared to IMR90-S cells (p=0.018) and significantly longer in Saos-2 compared to UMUC3 (p=0.028) (FIG. 14). The lack of consistently longer telomeres in ALT+ cells compared to all TEL+ and IMR90-S cells thus leads us to conclude that neither the overall telomere mean length nor the end telomere (−TFE) mean length are reliable readouts for differentiating ALT+ from TEL+ cells.

Here, the present study reported shorter overall mean telomere lengths in ALT+ cell lines. For example, U2OS overall telomere mean lengths reported using telomere combing assay (TCA) are between 35-45 kb while measurements with SMTA-OM are 3.4±5.1 kb (Table 1). Possible reasons for these discrepancies are as follows: First, the SMTA-OM can detect telomeres as short as 100 bp. The present study measured 2000 single molecule telomeres from the three ALT+ cell lines, 33% of which had telomeres measuring less than 800 bp. Out of the three ALT+ cell lines analyzed, U2OS had the most telomeres less than 800 bp at 44%, followed by SK-MEL-2 with 30%, and Saos-2 with 25%. The telomeres shorter than 800 bp are most likely missed by the TCA and terminal restriction fragment (TRF) assays. Second, the ability of SMTA-OM to detect signal loss from TFEs and ITS− will substantially decrease the overall mean telomere lengths in these ALT+ cell lines. Third, the patterns of the Nt. BspQI nick labeling can unambiguously identify the end of a chromosome arm. This makes it possible to distinguish the fusion/ITS+ from the interstitial telomere repeats found internally, which were excluded from the calculations in the TCA assay. Intriguingly, the telomeres in the fusion/ITS+ are much shorter than the terminal telomeres (Table 1). Fourth, the present study detected a higher percentage of fusion/ITS+ in the three ALT+ cells: U2OS (23.1%), Saos-2 (17.6%), and SK-MEL-2 (8.6%). These fusions/ITS+ likely render the telomeres in the TRF assay seemingly longer than they really are. Collectively, it is noted SMTA-OM is the best technology to define the features of chromosome ends and telomeres.

The much more prevalent super-long telomeres appear to be a unique feature of the ALT+ cells. The longest telomeres from the two TEL+ cells rarely exceed 10 kb. In contrast, telomeres with a length over 10 kb are easily detected in all three ALT+ cells. In U2OS, SK-MEL-2, and Saos-2, telomeres measuring longer than 10 kb accounted for 69%, 63%, and 43% of chromosome arms, respectively. In stark contrast, UMUC3 and LNCaP had telomeres greater than 10 kb for only 26% and 9% of arms, respectively, while 29% of arms had telomeres longer than 10 kb in IMR90-S cells. The longest telomeres detected in the three ALT+ cells are one molecule of the 16q of Saos-2, measuring at 62.8 kb, one molecule of the 7q of SK-MEL-2 measuring 47.3 kb, and one molecule of the 3p of U2OS measuring at 35.8 kb. The present study thus propose that the prevalence of super-long telomere can potentially be used to differentiate ALT+ from TEL+ cells.

The telomere lengths of individual chromosome arms vary substantially for both ALT+ and TEL+ cells. The SMTA-OM assay can accurately quantify the heterogeneity of telomere length because the telomere length of individual chromosome arms for most chromosomes can be measured at the single-molecule level. In ALT+ cells specifically, there are many super-long telomeres as well as telomeres measuring at 0 kb because of the fusion/ITS− and TFEs. The increased heterogeneity of the telomere length in ALT+ cells is reflected in higher standard deviations and higher CV values (FIG. 12). All three ALT+ cells have CV values greater than 1 and a much broader telomere length distribution compared to the two TEL+ cells and IMR90-S cells, which have a much tighter telomere length distribution and CV values less than one.

The percentage of the fusion/ITS+ is also a useful indicator for differentiating ALT+ from TEL+ cells. All three ALT+ cell lines manifest a high prevalence of fusion/ITS+, while no fusion/ITS+ was detected in the two TEL+ cell lines and IMR90-S. The reason for this dramatic difference may be due to heightened spontaneous telomeric damage in the ALT+ cells. When two damaged telomeres are repaired through various DSB repair pathways—for example, nonhomologous end joining (NHEJ) or HDR/BIR—it will lead to end-to-end chromosome fusions (i.e., the dicentric chromosomes), which then manifest as fusion/ITS+ in the SMTA-OM. Intriguingly, there also seem to be chromosome arm-specific effects of the fusion events. Chromosome arms that were analyzed in all three ALT+ cell lines and with detectable fusion/ITS+ include 5q, 7q, 17q, and 21q. There are no shared chromosome arms, of which the fusion/ITS+ is increased in all three ALT+ cells. Finally, there is no fusion/ITS+ detected for 14q in any of the three ALT+ cell lines.

The increase in TFEs is another unique characteristic of the three ALT+ cells, which can also potentially be used as an ALT+ identifier. Similar to fusion/ITS+, significant numbers of TFEs were observed in all three ALT+ cells, while none were detected in the two TEL+ cells and IMR90-S. Similar to the fusion/ITS+, the increased TFEs in the ALT+ cells are likely due to the heightened spontaneous DNA damages in the subtelomeres, which would result in the complete loss of telomeres. U2OS had the greatest elevation of TFEs, followed by SK-MEL-2, and Saos-2 (Table 1). For the individual chromosome arms where measurements could be conducted from U2OS, SK-MEL-2, and Saos-2, TFEs were absent only from 5q. TFEs were observed in all three ALT+ cells from chromosome arms: 1q, 2p, 8q, 9p, 12p, 14q, 16q, 18q, and 19p. Moreover, when two TFEs are repaired by the NHEJ, it would then produce a fusion/ITS− molecule. Alternatively, fusion/ITS− can also be generated through fusing a TFE with a double-stranded DNA break in the internal chromosomal region. Therefore, the data indicate that in addition to the heightened spontaneous DNA damage at telomeres, the subtelomeres of the ALT+ cells are also prone to spontaneous DNA damages.

ECTRs can be easily detected by SMTA-OM and are quite abundant in all three ALT+ cells (FIG. 9B). The number of ECTRs alone or in combination with other SMTA-OM readouts may be used to distinguish ALT+ cells from the TEL+ cells.

### Example 2-5

A workflow that simultaneously measures and analyzes telomere lengths while optically mapping the subtelomeric/chromosome end regions of long DNA molecules has been established. Three ALT+ cell lines (U2OS, SK-MEL-2, and Saos-2) and two TEL+ cell lines (UMUC3, and LNCaP), along with senescent IMR90, were analyzed for unique indicators for differentiating cancers based on their TMMs. The analysis of end telomere lengths, heterogeneity of telomere lengths, frequency of fusion/ITS+, fusion/ITS−, super-long telomeres, and TFEs reveals characteristics specific to ALT+ cells. Certain SMTA-OM readouts, alone or in combination with others, can reliably differentiate ALT+ cells from TEL+ cells.

In addition to comparing ALT+ and TEL+ cells, the present study has also observed significant telomere/chromosome end differences among the three ALT+ cells. The parameters investigated, such as telomere length heterogeneity, ITS+, TFE, and CV, seem to fluctuate even across the three ALT+ cell lines. Saos-2 and U2OS, overall, have similar trends in these parameters compared to SK-MEL-2. When the comparison is conducted at the level of individual chromosome arms, there are even more pronounced discrepancies among the three ALT+ cells. Therefore, these chromosome arm-specific changes detected by the SMTA-OM may also be used to differentiate ALT+ cancers.

FANCM has been identified as a promising molecular target for treating ALT+ cancers (Lu et al., Nat Commun. 2019; 10:2252; Silva et al., Nat. Commun. 2019; 10:2253; Pan et al, Proc. Natl. Acad. Sci. USA. 2017; 114:E5940-E5949). It has also been reported that the ALT+ cells are especially sensitive to the treatment of small molecule inhibitors targeting ATR (ATRi) and PARP (PARPi) (Zimmermann et al., Cell Rep. 2022; 40:111081). Four PARPi have been approved by the FDA to treat various BRCA1/2-deficient cancers (Wicks et al., Open Biol. 2022; 12:220118). A few ATRi are currently in various stages of clinical trials (da Costa et al., Nat. Rev. Drug Discov. 2023; 22:38-58; Cybulla et al., Nat. Rev. Cancer. 2022; 23:6-24). SMTA-OM can be used in the clinic as a diagnostic tool to identify ALT+ tumors for targeted cancer therapy.

### ENUMERATED EMBODIMENTS

In some aspects, the present invention is directed to the following non-limiting embodiments:

Embodiment 1: A method of categorizing a cancer, the method comprising: performing a global analysis of telomere lengths in cancer cells of the cancer, wherein:
(i) an increased abundance of fusions/internal telomere-like sequence (ITS+) in the cancer cells relative to a reference abundance indicates a cancer utilizing an alternative lengthening of telomere pathway (ALT+ cancer);
(ii) an increased abundance of fusions/internal telomere-like sequence loss (ITS−) in the cancer cells relative to a reference abundance indicates an ALT+ cancer;
(iii) an increased abundance of telomere-free ends (TFEs) in the cancer cells relative to a reference abundance indicates an ALT+ cancer;
(iv) an increased abundance of telomeres having 20% longer length than a natural telomere length (super-long telomeres) in the cancer cells relative to a reference abundance indicates an ALT+ cancer; and/or
(v) an increased level of heterogeneity of telomere lengths at the whole-genome level in the cancer cells relative to a reference level indicates an ALT+ cancer.

Embodiment 2: The method of Embodiment 1, wherein performing the global analysis of telomere lengths in the cancer cells comprises: contacting genomic DNA of the cancer cells with a guide RNA (gRNA) having a portion complementary to a telomere-specific motif, and a nickase to introduce a nick in the telomere; contacting the nicked DNA with a polymerase and a nucleotide labeled with a first dye such that the nucleotide labeled by the first dye is incorporated into the nicked telomere; and detecting the telomere using a signal of the first dye.

Embodiment 3: The method of Embodiment 2, wherein the gRNA guides the nikase to the telomere, thereby allowing the nickase to introduce the nick in the telomere.

Embodiment 4: The method of claim 2, wherein at least one of the following applies: (a) the gRNA is complementary to a telomere repeat (TTAGGG)n, and/or (b) the nikase is a Cas9 nickase.

Embodiment 5: The method of claim 2, further comprising: labeling the genomic DNA of the cancer cells with a second dye labeling the genomic DNA; and detecting the genomic DNA according to a signal of the second dye.

Embodiment 6: The method of Embodiment 2, wherein the first dye or the second dye is a fluorescent dye.

Embodiment 7: The method of Embodiment 2, further comprising: generating a recognition pattern on each chromosome of the cancer cells; and matching sections of the cancer cell chromosomes with chromosome sections of a healthy cell according to the recognition pattern, wherein generating the recognition pattern the chromosomes of the cancer cells comprises:
contacting the genomic DNA with a motif-specific nicking endonuclease, thereby producing a second nick in the genomic DNA at the motif sequence; and
contacting the nicked DNA with a polymerase and a nucleotide labeled with a third dye such that the nucleotide labeled with the third dye is incorporated into the nicked DNA at the motif sequence location.

Embodiment 8: The method of Embodiment 7, wherein the motif-specific nicking endonuclease is Nt.BspQI.

Embodiment 9: The method of Embodiment 7, wherein the third dye is the same as the first dye or the third dye produce a same signal as the first dye; or the third dye is different from the first dye or the third dye produce a different signal from that produced by the first dye.

Embodiment 10: The method of Embodiment 2, wherein detecting the telomere according to the signal of the first dye comprises analyzing the genomic DNA labeled with the first dye according to a nanochannel array method.

Embodiment 11: A method of treating cancer in a subject in need thereof, comprising: determining whether the cancer is a cancer having activated expression of telomerase (TEL+ cancer) or a cancer utilizing an alternative lengthening of telomere pathway (ALT+ cancer); and if the cancer is determined to be an ALT+ cancer, administering to the subject an effective amount of an inhibitor for Fanconi anemia, complementation group M (FANCM), an inhibitor for ataxia telangiectasia and Rad3-related (ATR), or an inhibitor for poly (ADP-ribose) polymerase (PARP), wherein determining whether the cancer a TEL+ cancer or an ALT+ cancer comprises performing a global analysis of telomere lengths in cancer cells of the cancer, and wherein:
(i) an increased abundance of fusions/internal telomere-like sequence (ITS+) in the cancer cells relative to a reference abundance indicates an ALT+ cancer;
(ii) an increased abundance of fusions/internal telomere-like sequence loss (ITS−) in the cancer cells relative to a reference abundance indicates an ALT+ cancer;
(iii) an increased abundance of telomere-free ends (TFEs) in the cancer cells relative to a reference abundance indicates an ALT+ cancer;
(iv) an increased abundance of telomeres having 20% longer length than a natural telomere length (super-long telomeres) in the cancer cells relative to a reference abundance indicates an ALT+ cancer; and/or
(v) an increased level of heterogeneity of telomere lengths at the whole-genome level in the cancer cells relative to a reference level indicates an ALT+ cancer.

Embodiment 12: The method of claim 11, wherein at least one of the following applies:
(a) the inhibitor for FANCM is at least one selected from the group consisting of MM2 peptide, and an RNA interference molecule targeting FANCM,
(b) the inhibitor for ATR is at least one selected from the group consisting of ART0380, ATG-018, ATRN-119, AZ20, berzosertib, Camonsertib (RP-3500), ceralasertib, CGK 733, dactolisib, elimusertib, ETP-46464, HAMNO (NSC-111847), IMP9064, SKLB-197, Schisandrin B, Torin 2, Tuvusertib, VE-821, VX-803 (M4344), and an RNA interference molecule targeting ATR,
(c) the inhibitor for PARP is at least one selected from the group consisting of 3-aminobenzamide, CEP 9722, E7016, iniparib, niraparib, olaparib, pamiparib, rucaparib, talazoparib, veliparib, and an RNA interference molecule targeting PARP.

Embodiment 13: The method of Embodiment 11, wherein performing the global analysis of telomere lengths in the cancer cells comprises:
contacting genomic DNA of the cancer cells with a guide RNA (gRNA) having a portion complementary to a telomere-specific motif, and a nickase to introduce a nick in the telomere;
contacting the nicked DNA with a polymerase and a nucleotide labeled with a first dye such that the nucleotide labeled by the first dye is incorporated into the nicked telomere; and detecting the telomere according to a signal of the first dye.

Embodiment 14: The method of Embodiment 13, wherein the gRNA guides the nikase to the telomere, thereby allowing the nickase to introduce the nick in the telomere.

Embodiment 15: The method of claim 13, wherein at least one of the following applies: (a) the gRNA is complementary to a telomere repeat (TTAGGG)n, and/or (b) the nikase is a Cas9 nickase.

Embodiment 16: The method of Embodiment 13, further comprising: labeling the genomic DNA of the cancer cells with a second dye labeling the genomic DNA; and detecting the genomic DNA according to a signal of the second dye.

Embodiment 17: The method of Embodiment 13, wherein the first dye or the second dye is a fluorescent dye.

Embodiment 18: The method of Embodiment 13, further comprising: generating a recognition pattern on each chromosome of the cancer cells; and matching sections of the cancer cell chromosomes with chromosome sections of a healthy cell according to the recognition pattern, wherein generating the recognition pattern the chromosomes of the cancer cells comprises:
contacting the genomic DNA with a motif-specific nicking endonuclease, thereby producing a second nick in the genomic DNA at the motif sequence; and
contacting the nicked DNA with a polymerase and a nucleotide labeled with a third dye such that the nucleotide labeled with the third dye is incorporated into the nicked DNA at the motif sequence location.

Embodiment 19: The method of Embodiment 18, wherein the motif-specific nicking endonuclease is Nt.BspQI.

Embodiment 20: The method of Embodiment 18, wherein the third dye is the same as the first dye or the third dye produce a same signal as the first dye; or the third dye is different from the first dye or the third dye produce a different signal from that produced by the first dye.

Embodiment 21: The method of Embodiment 13, wherein detecting the telomere according to the signal of the first dye comprises analyzing the genomic DNA labeled with the first dye according to a nanochannel array method.

Embodiment 22: The method of Embodiment 11, wherein the subject is a mammal or a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of categorizing a cancer, the method comprising:
performing a global analysis of telomere lengths in cancer cells of the cancer, wherein:
(i) an increased abundance of fusions/internal telomere-like sequence (ITS+) in the cancer cells relative to a reference abundance indicates a cancer utilizing an alternative lengthening of telomere pathway (ALT+ cancer);
(ii) an increased abundance of fusions/internal telomere-like sequence loss (ITS−) in the cancer cells relative to a reference abundance indicates an ALT+ cancer;
(iii) an increased abundance of telomere-free ends (TFEs) in the cancer cells relative to a reference abundance indicates an ALT+ cancer;
(iv) an increased abundance of telomeres having 20% longer length than a natural telomere length (super-long telomeres) in the cancer cells relative to a reference abundance indicates an ALT+ cancer; and/or
(v) an increased level of heterogeneity of telomere lengths at the whole-genome level in the cancer cells relative to a reference level indicates an ALT+ cancer.

2. The method of claim 1, wherein performing the global analysis of telomere lengths in the cancer cells comprises:
contacting genomic DNA of the cancer cells with a guide RNA (gRNA) having a portion complementary to a telomere-specific motif, and a nickase to introduce a nick in the telomere;
contacting the nicked DNA with a polymerase and a nucleotide labeled with a first dye such that the nucleotide labeled by the first dye is incorporated into the nicked telomere; and
detecting the telomere using a signal of the first dye.

3. The method of claim 2, wherein the gRNA guides the nikase to the telomere, thereby allowing the nickase to introduce the nick in the telomere.

4. The method of claim 2, wherein at least one of the following applies:
(a) the gRNA is complementary to a telomere repeat (TTAGGG)n, and/or
(b) the nikase is a Cas9 nickase.

5. The method of claim 2, further comprising:
labeling the genomic DNA of the cancer cells with a second dye labeling the genomic DNA; and
detecting the genomic DNA according to a signal of the second dye.

6. The method of claim 2, wherein the first dye or the second dye is a fluorescent dye.

7. The method of claim 2, further comprising:
generating a recognition pattern on each chromosome of the cancer cells; and
matching sections of the cancer cell chromosomes with chromosome sections of a healthy cell according to the recognition pattern,
wherein generating the recognition pattern the chromosomes of the cancer cells comprises:
contacting the genomic DNA with a motif-specific nicking endonuclease, thereby producing a second nick in the genomic DNA at the motif sequence; and
contacting the nicked DNA with a polymerase and a nucleotide labeled with a third dye such that the nucleotide labeled with the third dye is incorporated into the nicked DNA at the motif sequence location.

8. The method of claim 7, wherein the motif-specific nicking endonuclease is Nt.BspQI.

9. The method of claim 7, wherein
the third dye is the same as the first dye or the third dye produce a same signal as the first dye; or
the third dye is different from the first dye or the third dye produce a different signal from that produced by the first dye.

10. The method of claim 2, wherein detecting the telomere according to the signal of the first dye comprises analyzing the genomic DNA labeled with the first dye according to a nanochannel array method.

11. A method of treating cancer in a subject in need thereof, comprising:
determining whether the cancer is a cancer having activated expression of telomerase (TEL+ cancer) or a cancer utilizing an alternative lengthening of telomere pathway (ALT+ cancer); and
if the cancer is determined to be an ALT+ cancer, administering to the subject an effective amount of an inhibitor for Fanconi anemia, complementation group M (FANCM), an inhibitor for ataxia telangiectasia and Rad3-related (ATR), or an inhibitor for poly (ADP-ribose) polymerase (PARP),
wherein determining whether the cancer a TEL+ cancer or an ALT+ cancer comprises performing a global analysis of telomere lengths in cancer cells of the cancer, and
wherein:
(i) an increased abundance of fusions/internal telomere-like sequence (ITS+) in the cancer cells relative to a reference abundance indicates an ALT+ cancer;
(ii) an increased abundance of fusions/internal telomere-like sequence loss (ITS−) in the cancer cells relative to a reference abundance indicates an ALT+ cancer;
(iii) an increased abundance of telomere-free ends (TFEs) in the cancer cells relative to a reference abundance indicates an ALT+ cancer;
(iv) an increased abundance of telomeres having 20% longer length than a natural telomere length (super-long telomeres) in the cancer cells relative to a reference abundance indicates an ALT+ cancer; and/or
(v) an increased level of heterogeneity of telomere lengths at the whole-genome level in the cancer cells relative to a reference level indicates an ALT+ cancer.

12. The method of claim 11, wherein at least one of the following applies:
(a) the inhibitor for FANCM is at least one selected from the group consisting of MM2 peptide, and an RNA interference molecule targeting FANCM,
(b) the inhibitor for ATR is at least one selected from the group consisting of ART0380, ATG-018, ATRN-119, AZ20, berzosertib, Camonsertib (RP-3500), ceralasertib, CGK 733, dactolisib, elimusertib, ETP-46464, HAMNO (NSC-111847), IMP9064, SKLB-197, Schisandrin B, Torin 2, Tuvusertib, VE-821, VX-803 (M4344), and an RNA interference molecule targeting ATR,
(c) the inhibitor for PARP is at least one selected from the group consisting of 3-aminobenzamide, CEP 9722, E7016, iniparib, niraparib, olaparib, pamiparib, rucaparib, talazoparib, veliparib, and an RNA interference molecule targeting PARP.

13. The method of claim 11, wherein performing the global analysis of telomere lengths in the cancer cells comprises:
contacting genomic DNA of the cancer cells with a guide RNA (gRNA) having a portion complementary to a telomere-specific motif, and a nickase to introduce a nick in the telomere;
contacting the nicked DNA with a polymerase and a nucleotide labeled with a first dye such that the nucleotide labeled by the first dye is incorporated into the nicked telomere; and
detecting the telomere according to a signal of the first dye.

14. The method of claim 13, wherein the gRNA guides the nikase to the telomere, thereby allowing the nickase to introduce the nick in the telomere.

15. The method of claim 13, wherein at least one of the following applies:
(a) the gRNA is complementary to a telomere repeat (TTAGGG)n, and/or
(b) the nikase is a Cas9 nickase.

16. The method of claim 13, further comprising:
labeling the genomic DNA of the cancer cells with a second dye labeling the genomic DNA; and
detecting the genomic DNA according to a signal of the second dye.

17. The method of claim 13, wherein the first dye or the second dye is a fluorescent dye.

18. The method of claim 13, further comprising:
generating a recognition pattern on each chromosome of the cancer cells; and
matching sections of the cancer cell chromosomes with chromosome sections of a healthy cell according to the recognition pattern,
wherein generating the recognition pattern the chromosomes of the cancer cells comprises:
contacting the genomic DNA with a motif-specific nicking endonuclease, thereby producing a second nick in the genomic DNA at the motif sequence; and
contacting the nicked DNA with a polymerase and a nucleotide labeled with a third dye such that the nucleotide labeled with the third dye is incorporated into the nicked DNA at the motif sequence location.

19. The method of claim 18, wherein the motif-specific nicking endonuclease is Nt.BspQI.

20. The method of claim 18, wherein
the third dye is the same as the first dye or the third dye produce a same signal as the first dye; or
the third dye is different from the first dye or the third dye produce a different signal from that produced by the first dye.

21. The method of claim 13, wherein detecting the telomere according to the signal of the first dye comprises analyzing the genomic DNA labeled with the first dye according to a nanochannel array method.

22. The method of claim 11, wherein the subject is a mammal or a human.


SEQUENCE LISTING (5 sequences)

SEQ ID NO: 1 - DNA, 16 bp, synthetic construct
  source 1..16: mol_type = other DNA
atgctcttcngtgatc

SEQ ID NO: 2 - DNA, 16 bp, synthetic construct
  source 1..16: mol_type = other DNA
gatcacngaagagcat

SEQ ID NO: 3 - DNA, 38 bp, synthetic construct
  source 1..38: mol_type = other DNA
ggttagggttagggttagggttagggttagggttaggg

SEQ ID NO: 4 - DNA, 38 bp, synthetic construct
  source 1..38: mol_type = other DNA
ccctaaccctaaccctaaccctaaccctaaccctaacc

SEQ ID NO: 5 - RNA, 20 nt, synthetic construct
  source 1..20: mol_type = other RNA
ttagggttagggttagggtt